SEQUENCE LISTING (24 sequences)

SEQ ID NO: 1 - DNA, 23 bp, Artificial Sequence
  primer
tgacgcacctgaagataactttg

SEQ ID NO: 2 - DNA, 25 bp, Artificial Sequence
  primer
gcacagaaatgttggtatgtgaaga

SEQ ID NO: 3 - DNA, 17 bp, Artificial Sequence
  primer
cggccacccatctgtca

SEQ ID NO: 4 - DNA, 21 bp, Artificial Sequence
  primer
tccagctgttacccaccaact

SEQ ID NO: 5 - DNA, 20 bp, Artificial Sequence
  primer
ttgctcagagcggagaaagc

SEQ ID NO: 6 - DNA, 20 bp, Artificial Sequence
  primer
cttgcaacgcgagtctgtgt

SEQ ID NO: 7 - DNA, 23 bp, Artificial Sequence
  primer
gggtggactaactttggacacaa

SEQ ID NO: 8 - DNA, 21 bp, Artificial Sequence
  primer
gaaatgcttcccagaccaaca

SEQ ID NO: 9 - DNA, 22 bp, Artificial Sequence
  primer
ccaagaatggaggctgtaggaa

SEQ ID NO: 10 - DNA, 25 bp, Artificial Sequence
  primer
ggattcagctgacttagcctatgag

SEQ ID NO: 11 - DNA, 22 bp, Artificial Sequence
  primer
ggctcctgtgaaaaagcttgtg

SEQ ID NO: 12 - DNA, 20 bp, Artificial Sequence
  primer
ggcagcatccatgattccat

SEQ ID NO: 13 - DNA, 19 bp, Artificial Sequence
  primer
atgggagcccacggaacta

SEQ ID NO: 14 - DNA, 20 bp, Artificial Sequence
  primer
aaccacccctcctgaaaggt

SEQ ID NO: 15 - DNA, 19 bp, Artificial Sequence
  primer
ccacggatgcctcaagaga

SEQ ID NO: 16 - DNA, 24 bp, Artificial Sequence
  primer
ccacagaaaaaaggagctgaaatt

SEQ ID NO: 17 - DNA, 20 bp, Artificial Sequence
  primer
agccttgccacaatctttgc

SEQ ID NO: 18 - DNA, 19 bp, Artificial Sequence
  primer
gtggaccggccctatgact

SEQ ID NO: 19 - DNA, 19 bp, Artificial Sequence
  primer
gagcccacctgccatcact

SEQ ID NO: 20 - DNA, 21 bp, Artificial Sequence
  primer
ctattgagccgagtccggaat

SEQ ID NO: 21 - DNA, 19 bp, Artificial Sequence
  primer
agagcccagagccgagatg

SEQ ID NO: 22 - DNA, 17 bp, Artificial Sequence
  primer
acgctgcccagcacgta

SEQ ID NO: 23 - DNA, 19 bp, Artificial Sequence
  primer
tgggcggagttaggaaagc

SEQ ID NO: 24 - DNA, 20 bp, Artificial Sequence
  primer
ggaactcggcctgacagatg

(19) United States
(12) Patent Application Publication
(10) Pub. No.: US 2012/0100999 A1
(43) Pub. Date: Apr. 26, 2012

(54) PROGNOSTIC GENE EXPRESSION SIGNATURE FOR SQUAMOUS CELL CARCINOMA OF THE LUNG

(75) Inventors: Ming-Sound Tsao, Toronto (CA); Chang-Qi Zhu, Aurora (CA); Igor Jurisica, Toronto (CA); Sandy D. Der, Toronto (CA); Frances A. Shepherd, Toronto (CA)

(73) Assignee: UNIVERSITY HEALTH NETWORK, Toronto (CA)

(21) Appl. No.: 13/265,534

(22) PCT Filed: Apr. 20, 2010
(86) PCT No.: PCT/CA2010/000596

§ 371 Date: Dec. 23, 2011

Related U.S. Application Data
(60) Provisional application No. 61/170,743, filed on Apr. 20, 2009.

(51) Int. Cl. C40B 30/00; G01N 33/566; C40B 40/06; C12Q 1/68

(52) U.S. Cl. 506/7; 435/6.12; 436/501; 435/7.1; 506/16

(57) ABSTRACT

Provided is a gene expression signature consisting of 12 biomarkers for use in prognosing or classifying a subject with lung squamous cell carcinoma into a poor survival group or a good survival group. The 12-gene signature specific for squamous cell carcinoma consists of the biomarkers RPL22, VEGFA, G0S2, NES, TNFRSF25, DKFZP586P0123, COL8A2, ZNF3, PJPK5, RNFT2, ARHGEF12 and PTPN20A.

### FIELD OF THE INVENTION

The application relates generally to methods for identifying biomarkers and biomarkers for squamous cell carcinoma of the lung.

### BACKGROUND OF THE INVENTION

Identifying gene expression signatures that capture altered key pathways/regulators in carcinogenesis may discover molecular subclasses and predict patient outcomes (1). Several prognostic gene expression signatures have been published for non-small cell lung cancer (NSCLC) (2-8) and its adenocarcinoma (ADC) subtype (9-12). Few studies have been performed to identify prognostic signatures specific for lung squamous cell carcinoma (SQCC) (13, 14), but their validation in independent cohorts or datasets has been limited.

Factors such as patient/sample heterogeneity, small sample size, variation in microarray platforms, RNA preparation and hybridization protocols could all contribute to difficulties in validation of gene expression signatures. In addition, the loss of information through arbitrary exclusion of patients or genes prior to analysis may play an important role. Supervised data mining methodology assigns cases into good and poor prognosis subgroups at specified time points (13, 15). This arbitrary assignment of a cutoff to split good/poor prognosis cases could be problematic due to the non-linear relationships between gene expression and patient survival. Other investigators have compared two extremes in outcome (very early death versus long survival) (3, 12); however, this approach may result in significant information loss, for almost half of the cases with intermediate survival are excluded from analysis, thereby leading to high finite sample variation (16), and making the cohort under study less representative. Therefore, it is anticipated that the validation of the identified signature could be very challenging.

It is estimated that most tissues express only 30-40% of genes (17) or 10,000 to 15,000 genes (18). Furthermore, among the expressed genes from similar tissue types, only a small fraction is differentially expressed. Only these differentially expressed genes distinguish one phenotype from another. In an attempt to compensate for this in genome-wide microarray studies, some investigators have excluded genes with low expression or low variation prior to signature selection (3, 8-10). This approach may result in the exclusion of potentially important low expression but key regulatory genes, leading to another potential source of information loss. In addition, signatures are generated using a forced forward inclusion procedure pre-determined by the rank of significance of the gene (8, 9) or the bootstrap score (13), regardless of whether the included gene contributes to the classification ability of the signature. The lack of heuristic measures in these methods potentially reduces the robustness of these signatures.

### SUMMARY OF THE INVENTION

According to a further aspect, there is provided a method of predicting prognosis in a subject with lung squamous cell carcinoma (SQCC) comprising the steps:
(a) obtaining a subject biomarker expression profile in a sample of the subject;
(b) obtaining a biomarker reference expression profile associated with a prognosis, wherein the subject biomarker expression profile and the biomarker reference expression profile each have values representing the expression level of at least one biomarker selected from RPL22, VEGFA, G0S2, NES, TNFRSF25, DKFZP586P0123, COL8A2, ZNF3, RIPK5, RNFT2, ARHGEF12 and PTPN20A;
(c) selecting the biomarker reference expression profile most similar to the subject biomarker expression profile, to thereby predict a prognosis for the subject.

According to a further aspect, there is provided a method of selecting a therapy for a subject with SQCC, comprising the steps:
(a) classifying the subject with SQCC into a poor survival group or a good survival group according to the method of any one of claims 1-19; and
(b) selecting adjuvant chemotherapy for the poor survival group or no adjuvant chemotherapy for the good survival group.

According to a further aspect, there is provided a method of selecting a therapy for a subject with SQCC, comprising the steps:
(a) determining the expression of at least one biomarker in a test sample from the subject selected from RPL22, VEGFA, G0S2, NES, TNFRSF25, DKFZP586P0123, COL8A2, ZNF3, RIPK5, RNFT2, ARHGEF12 and PTPN20A;
(b) comparing the expression of the at least one biomarker in the test sample with the same biomarker in a control sample;
(c) classifying the subject in a poor survival group or a good survival group, wherein a difference or a similarity in the expression of the at least three biomarkers between the control sample and the test sample is used to classify the subject into a poor survival group or a good survival group;
(d) selecting adjuvant chemotherapy if the subject is classified in the poor survival group and selecting no adjuvant chemotherapy if the subject is classified in the good survival group.

According to a further aspect, there is provided a composition comprising a plurality of isolated nucleic acid sequences, wherein each isolated nucleic acid sequence hybridizes to:
(e) a RNA product of at least one of twelve genes: RPL22, VEGFA, G0S2, NES, TNFRSF25, DKFZP586P0123, COL8A2, ZNF3, RIPK5, RNFT2, ARHGEF12 and PTPN20A; and/or
(f) a nucleic acid complementary to a),
wherein the composition is used to measure the level of RNA expression of the genes.

According to a further aspect, there is provided an array comprising, for each of at least one of twelve genes: RPL22, VEGFA, G0S2, NES, TNFRSF25, DKFZP586P0123, COL8A2, ZNF3, RIPK5, RNFT2, ARHGEF12 and PTPN20A, one or more polynucleotide probes complementary and hybridizable to an expression product of the gene.

According to a further aspect, there is provided a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out a method described herein.

According to a further aspect, there is provided a computer implemented product for predicting a prognosis or classifying a subject with SQCC comprising:
(a) a means for receiving values corresponding to a subject expression profile in a subject sample; and
(b) a database comprising a reference expression profile associated with a prognosis, wherein the subject biomarker expression profile and the biomarker reference profile each have at least three values representing the expression level of at least one biomarker selected from RPL22, VEGFA, G0S2, NES, TNFRSF25, DKFZP586P0123, COL8A2, ZNF3, RIPK5, RNFT2, ARHGEF12 and PTPN20A;
wherein the computer implemented product selects the biomarker reference expression profile most similar to the subject biomarker expression profile, to thereby predict a prognosis or classify the subject.

According to a further aspect, there is provided a computer implemented product for determining therapy for a subject with SQCC comprising:
(a) a means for receiving values corresponding to a subject expression profile in a subject sample; and
(b) a database comprising a reference expression profile associated with a therapy, wherein the subject biomarker expression profile and the biomarker reference profile each have at least one value, the at least one value representing the expression level of at least one biomarker selected from RPL22, VEGFA, G0S2, NES, TNFRSF25, DKFZP586P0123, COL8A2, ZNF3, RIPK5, RNFT2, ARHGEF12 and PTPN20A;
wherein the computer implemented product selects the biomarker reference expression profile most similar to the subject biomarker expression profile, to thereby predict the therapy.

According to a further aspect, there is provided a computer implemented product described herein for use with a method described herein.

According to a further aspect, there is provided a computer readable medium having stored thereon a data structure for storing a computer implemented product described herein.

According to a further aspect, there is provided a computer system comprising
(a) a database including records comprising a biomarker reference expression profile of at least one gene selected from RPL22, VEGFA, G0S2, NES, TNFRSF25, DKFZP586P0123, COL8A2, ZNF3, RIPK5, RNFT2, ARHGEF12 and PTPN20A associated with a prognosis or therapy;
(b) a user interface capable of receiving a selection of gene expression levels of the at least one gene for use in comparing to the biomarker reference expression profile in the database;
(c) an output that displays a prediction of prognosis or therapy according to the biomarker reference expression profile most similar to the expression levels of the at least one gene.

According to a further aspect, there is provided a kit to prognose or classify a subject with early stage SQCC, comprising detection agents that can detect the expression products of at least one biomarker selected from RPL22, VEGFA, G0S2, NES, TNFRSF25, DKFZP586P0123, COL8A2, ZNF3, RIPK5, RNFT2, ARHGEF12 and PTPN20A, and instructions for use.

According to a further aspect, there is provided a kit to select a therapy for a subject with SQCC, comprising detection agents that can detect the expression products of at least one biomarker selected from RPL22, VEGFA, G0S2, NES, TNFRSF25, DKFZP586P0123, COL8A2, ZNF3, RIPK5, RNFT2, ARHGEF12 and PTPN20A, and instructions for use.

### BRIEF DESCRIPTION OF THE DRAWINGS

These and other features of the preferred embodiments of the invention will become more apparent in the following detailed description in which reference is made to the appended drawings wherein:

FIG. 1 shows selection of the prognostic signature. A: Pipeline of the identification and validation of the prognostic signature. Ninety-six probe sets from 19,619 probe sets with Grade A annotations were pre-selected by univariate analysis at p<0.005. The signature was selected sequentially by exclusion and inclusion procedures. B: Plot of the exclusion/inclusion selection. C: Survival curves of the low and high risk groups classified by the 12-gene signature in the training set

FIG. 2 shows in silico and qPCR validation of the 12-gene signature in SQCC samples from Duke (A-C), SKKU (D-F) and UHN (G-I). Note: Recurrence-free survival was used for SKKU.

FIG. 3 shows genes of the 12-gene signature, Sun 50-gene, and Raponi 50-gene SQCC prognostic signatures mapped to protein-protein interaction (PPI) data form a connected PPI network. Genes of the 12-gene and two previously published prognostic signatures for SQCC were mapped to protein-protein interaction (PPI) data in I2D (v.1.7; http::Rophid.utoronto.ca/i2d) and visualized in NaVIGaTOR v.2.08 (http::Rophid.utoronto.ca/navigator) (24). The network comprises of 1,075 proteins and 14,651 interactions. Shapes/nodes represent proteins and lines/edges are indicating interactions. Node color corresponds to biological function according to Gene Ontology (GO) annotation as indicated in the legend. The 12-gene signature, 8 out of 12 genes were mapped to PPI data. Sun 50-gene signature, 31 of 42 targets were mapped. Raponi 50-gene signature, 35 of 48 targets were mapped. Eight out of 9 genes overlapping between Sun 50-gene and Raponi 50-gene signatures were mapped to PPI data. Direct interaction between the 12-gene signature gene ARHGEF12 and IGF1R, a therapeutic target in SQCC, is indicated by turquoise edge color (top right). Faded-out nodes and edges correspond to interactions of individual signature genes, which do not contribute to the interaction between the 3 signatures.

FIGS. 4 shows Kaplan-Meier curves of the 12-gene signature in ADC patients from the 3 validation sets (A-C).

### DETAILED DESCRIPTION

The application generally relates to identifying gene signatures and provides methods and computer implemented products therefore. The application also relates to 12 biomarkers that form 1-gene to 12-gene signatures, and provides methods, compositions, computer implemented products, detection agents and kits for prognosing or classifying a subject with SQCC and for determining the benefit of adjuvant chemotherapy.

Global gene expression profiling has been implemented successfully for tumor characterization, classification and prediction of disease outcome. However, few studies have explored prognostic signatures for squamous cell carcinoma of the lung (SQCC).

A published microarray dataset from 129 SQCC patients was used as a training set to identify the minimal gene set prognostic signature. This was selected using the MAximizing R Square Algorithm (MARSA), a novel heuristic signature optimization procedure based on goodness-of-fit (R square). The signature was tested internally by leave-one-out-cross-validation (LOOCV), and then externally in 3 independent public lung cancer microarray datasets: 2 datasets of NSCLC and one of adenocarcinoma (ADC) only. Quantitative-PCR (QPCR) was used to validate the signature in a fourth independent SQCC cohort.

A 12-gene signature that passed the internal LOOCV validation was identified. The signature was independently prognostic for SQCC in two NSCLC datasets (total n=223) but not in ADC. The lack of prognostic significance in ADC was confirmed in the largest available ADC dataset (n=442). The prognostic significance of the signature was validated further by qPCR in another independent cohort containing 62 SQCC samples (HR=3.76, 95% CI 1.10-12.87, p=0.035).

We have identified a novel 12-gene prognostic signature specific for SQCC and demonstrated the effectiveness of MARSA to identify prognostic gene expression signatures.

It must be noted that as used herein and in the appended claims, the singular forms “a”, “an” and “the” include the plural referents unless the context clearly dictates otherwise.

As used herein, “biological parameter” may refer to any measurable or quantifiable characteristic in a biological system and includes, without limitation, physical characteristics and attributes, genotype, phenotype, biomarkers, gene expression, splice-variants of an mRNA, polymorphisms of DNA or protein, levels of protein, cells, nucleic acids, amino acids or other biological matter.

The term “biomarker” as used herein refers to a gene that is differentially expressed in individuals. For example, specifically with respect to lung squamous cell carcinoma (SQCC), the biomarkers may be differentially expressed in individuals according to prognosis and thus may be predictive of different survival outcomes and of the benefit of adjuvant chemotherapy. In one embodiment, the 12 biomarkers that form the SQCC gene signature of the present application are RPL22, VEGFA, G0S2, NES, TNFRSF25, DKFZP586P0123, COL8A2, ZNF3, RIPK5, RNFT2, ARHGEF12 and PTPN20A.

The term “level of expression” or “expression level” as used herein refers to a measurable level of expression of the products of biomarkers, such as, without limitation, the level of messenger RNA transcript expressed or of a specific exon or other portion of a transcript, the level of proteins or portions thereof expressed of the biomarkers, the number or presence of DNA polymorphisms of the biomarkers, the enzymatic or other activities of the biomarkers, and the level of specific metabolites.

The term “reference expression profile” as used herein refers to the expression level of at least one of the 12 biomarkers selected from RPL22, VEGFA, G0S2, NES, TNFRSF25, DKFZP586P0123, COL8A2, ZNF3, RIMS, RNFT2, ARHGEF12 and PTPN20A associated with a clinical outcome in a SQCC patient. The reference expression profile comprises up to 12 values, each value representing the level of a biomarker, wherein each biomarker corresponds to one gene selected from RPL22, VEGFA, G0S2, NES, TNFRSF25, DKFZP586P0123, COL8A2, ZNF3, RIPK5, RNFT2, ARHGEF12 and PTPN20A. The reference expression profile is typically identified using one or more samples comprising tumor or adjacent or other-wise tumour-related stromal/blood based tissue or cells, wherein the expression is similar between related samples defining an outcome class or group such as poor survival or good survival and is different to unrelated samples defining a different outcome class such that the reference expression profile is associated with a particular clinical outcome. The reference expression profile is accordingly a reference profile or reference signature of the expression of at least 1 of the 12 biomarkers selected from RPL22, VEGFA, G0S2, NES, TNFRSF25, DKFZP586P0123, COL8A2, ZNF3, RIPK5, RNFT2, ARHGEF12 and PTPN20A, to which the subject expression levels of the corresponding genes in a patient sample are compared in methods for determining or predicting clinical outcome.

As used herein, the term “control” refers to a specific value or dataset that can be used to prognose or classify the value e.g expression level or reference expression profile obtained from the test sample associated with an outcome class. In one embodiment, a dataset may be obtained from samples from a group of subjects known to have SQCC and good survival outcome or known to have SQCC and have poor survival outcome or known to have SQCC and have benefited from adjuvant chemotherapy or known to have SQCC and not have benefited from adjuvant chemotherapy. The expression data of the biomarkers in the dataset can be used to create a control value that is used in testing samples from new patients. In such an embodiment, the “control” is a predetermined value for the set of at least 1 of the 12 biomarkers obtained from SQCC patients whose biomarker expression values and survival times are known. Alternatively, the “control” is a predetermined reference profile for the set of at least three of the sixteen biomarkers described herein obtained from patients whose survival times are known.

A person skilled in the art will appreciate that the comparison between the expression of the biomarkers in the test sample and the expression of the biomarkers in the control will depend on the control used. For example, if the control is from a subject known to have SQCC and poor survival, and there is a difference in expression of the biomarkers between the control and test sample, then the subject can be prognosed or classified in a good survival group. If the control is from a subject known to have SQCC and good survival, and there is a difference in expression of the biomarkers between the control and test sample, then the subject can be prognosed or classified in a poor survival group. For example, if the control is from a subject known to have SQCC and good survival, and there is a similarity in expression of the biomarkers between the control and test sample, then the subject can be prognosed or classified in a good survival group. For example, if the control is from a subject known to have SQCC and poor survival, and there is a similarity in expression of the biomarkers between the control and test sample, then the subject can be prognosed or classified in a poor survival group.

The term “differentially expressed” or “differential expression” as used herein refers to a difference in the level of expression of the biomarkers that can be assayed by measuring the level of expression of the products of the biomarkers, such as the difference in level of messenger RNA transcript or a portion thereof expressed or of proteins expressed of the biomarkers. In a preferred embodiment, the difference is statistically significant. The term “difference in the level of expression” refers to an increase or decrease in the measurable expression level of a given biomarker, for example as measured by the amount of messenger RNA transcript and/or the amount of protein in a sample as compared with the measurable expression level of a given biomarker in a control. In one embodiment, the differential expression can be compared using the ratio of the level of expression of a given biomarker or biomarkers as compared with the expression level of the given biomarker or biomarkers of a control, wherein the ratio is not equal to 1.0. For example, an RNA or protein is differentially expressed if the ratio of the level of expression in a first sample as compared with a second sample is greater than or less than 1.0. For example, a ratio of greater than 1, 1.2, 1.5, 1.7, 2, 3, 3, 5, 10, 15, 20 or more, or a ratio less than 1, 0.8, 0.6, 0.4, 0.2, 0.1, 0.05, 0.001 or less. In another embodiment the differential expression is measured using p-value. For instance, when using p-value, a biomarker is identified as being differentially expressed as between a first sample and a second sample when the p-value is less than 0.1, preferably less than 0.05, more preferably less than 0.01, even more preferably less than 0.005, the most preferably less than 0.001.

The term “similarity in expression” as used herein means that there is no or little difference in the level of expression of the biomarkers between the test sample and the control or reference profile. For example, similarity can refer to a fold difference compared to a control. In a preferred embodiment, there is no statistically significant difference in the level of expression of the biomarkers.

The term “most similar” in the context of a reference profile refers to a reference profile that is associated with a clinical outcome that shows the greatest number of identities and/or degree of changes with the subject profile.

The term “prognosis” as used herein refers to a clinical outcome group such as a poor survival group or a good survival group associated with a disease subtype which is reflected by a reference profile such as a biomarker reference expression profile or reflected by an expression level of the biomarkers disclosed herein. The prognosis provides an indication of disease progression and includes an indication of likelihood of death due to lung cancer. In one embodiment the clinical outcome class includes a good survival group and a poor survival group.

The term “prognosing or classifying” as used herein means predicting or identifying the clinical outcome group that a subject belongs to according to the subject's similarity to a reference profile or biomarker expression level associated with the prognosis. For example, prognosing or classifying comprises a method or process of determining whether an individual with SQCC has a good or poor survival outcome, or grouping an individual with SQCC into a good survival group or a poor survival group, or predicting whether or not an individual with SQCC will respond to therapy.

The term “good survival” as used herein refers to an increased chance of survival as compared to patients in the “poor survival” group. For example, the biomarkers of the application can prognose or classify patients into a “good survival group”. These patients are at a lower risk of death after surgery.

The term “poor survival” as used herein refers to an increased risk of death as compared to patients in the “good survival” group. For example, biomarkers or genes of the application can prognose or classify patients into a “poor survival group”. These patients are at greater risk of death or adverse reaction from disease or surgery, treatment for the disease or other causes.

The term “subject” as used herein refers to any member of the animal kingdom, preferably a human being and most preferably a human being that has SQCC or that is suspected of having SQCC.

The term “test sample” as used herein refers to any fluid, cell or tissue sample from a subject which can be assayed for biomarker expression products and/or a reference expression profile, e.g. genes differentially expressed in subjects with SQCC according to survival outcome.

The phrase “determining the expression of biomarkers” as used herein refers to determining or quantifying RNA or proteins or protein activities or protein-related metabolites expressed by the biomarkers. The term “RNA” includes mRNA transcripts, and/or specific spliced or other alternative variants of mRNA, including anti-sense products. The term “RNA product of the biomarker” as used herein refers to RNA transcripts transcribed from the biomarkers and/or specific spliced or alternative variants. In the case of “protein”, it refers to proteins translated from the RNA transcripts transcribed from the biomarkers. The term “protein product of the biomarker” refers to proteins translated from RNA products of the biomarkers.

A person skilled in the art will appreciate that a number of methods can be used to detect or quantify the level of RNA products of the biomarkers within a sample, including arrays, such as microarrays, RT-PCR (including quantitative RT-PCR), nuclease protection assays and Northern blot analyses.

Accordingly, in one embodiment, the biomarker expression levels are determined using arrays, optionally microarrays, RT-PCR, optionally quantitative RT-PCR, nuclease protection assays or Northern blot analyses.

In another embodiment, the biomarker expression levels are determined by using an array.

In one embodiment, the array is a HG-U133A chip from Affymetrix. In another embodiment, a plurality of nucleic acid probes that are complementary or hybridizable to an expression product of at least one of the 12 biomarkers selected from RPL22, VEGFA, G0S2, NES, TNFRSF25, DKFZP586P0123, COL8A2, ZNF3, RIPK5, RNFT2, ARHGEF12 and PTPN20A are used on the array.

The term “nucleic acid” includes DNA and RNA and can be either double stranded or single stranded.

The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The term “probe” as used herein refers to a nucleic acid sequence that will hybridize to a nucleic acid target sequence. In one example, the probe hybridizes to an RNA product of the biomarker or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

In another embodiment, the biomarker expression levels are determined by using quantitative RT-PCR. In another embodiment, the primers used for quantitative RT-PCR comprise a forward and reverse primer for each of RPL22, VEGFA, G0S2, NES, TNFRSF25, DKFZP586P0123, COL8A2, ZNF3, RIPK5, RNFT2, ARHGEF12 and PTPN20A.

The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less or more. The factors involved in determining the appropriate length of primer are readily known to one of ordinary skill in the art.

In addition, a person skilled in the art will appreciate that a number of methods can be used to determine the amount of a protein product of the biomarker of the invention, including immunoassays such as Western blots, ELISA, and immunoprecipitation followed by SDS-PAGE and immunocytochemistry.

Accordingly, in another embodiment, an antibody is used to detect the polypeptide products of at least 1 of the 12 biomarkers selected from RPL22, VEGFA, G0S2, NES, TNFRSF25, DKFZP586P0123, COL8A2, ZNF3, RIPK5, RNFT2, ARHGEF12 and PTPN20A. In another embodiment, the sample comprises a tissue sample. In a further embodiment, the tissue sample is suitable for immunohistochemistry.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)2, scFv, dsFv, ds-scFv, dimers, minibodies, diabodies, and multimers thereof and bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

Conventional techniques of molecular biology, microbiology and recombinant DNA techniques are within the skill of the art. Such techniques are explained fully in the literature. See, e.g., Sambrook, Fritsch & Maniatis, 1989, Molecular Cloning: A Laboratory Manual, Second Edition; Oligonucleotide Synthesis (M. J. Gait, ed., 1984); Nucleic Acid Hybridization (B. D. Harnes & S. J. Higgins, eds., 1984); A Practical Guide to Molecular Cloning (B. Perbal, 1984); and a series, Methods in Enzymology (Academic Press, Inc.); Short Protocols In Molecular Biology, (Ausubel et al., ed., 1995).

For example, antibodies having specificity for a specific protein, such as the protein product of a biomarker, may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

The gene signature described herein can be used to select treatment for SQCC patients. As explained herein, the biomarkers can classify patients with SQCC into a poor survival group or a good survival group and into groups that might benefit from adjuvant chemotherapy or not.

The term “adjuvant chemotherapy” as used herein means treatment of cancer with chemotherapeutic agents after surgery where all detectable disease has been removed, but where there still remains a risk of small amounts of remaining cancer. Typical chemotherapeutic agents include cisplatin, carboplatin, vinorelbine, gemcitabine, doccetaxel, paclitaxel and navelbine.

According to one aspect, there is provided a method of prognosing or classifying a subject with lung squamous cell carcinoma SQCC comprising:
(a) determining the expression of at least one biomarker in a test sample from the subject selected from RPL22, VEGFA, G0S2, NES, TNFRSF25, DKFZP586P0123, COL8A2, ZNF3, RIPK5, RNFT2, ARHGEF12 and PTPN20A; and
(b) comparing expression of the at least one biomarker in the test sample with expression of the at least one biomarker in a control sample;
wherein a difference or similarity in the expression of the at least one biomarker between the control and the test sample is used to prognose or classify the subject with SQCC into a poor survival group or a good survival group.

According to a further aspect, there is provided a method of predicting prognosis in a subject with lung squamous cell carcinoma (SQCC) comprising the steps:
(a) obtaining a subject biomarker expression profile in a sample of the subject;
(b) obtaining a biomarker reference expression profile associated with a prognosis, wherein the subject biomarker expression profile and the biomarker reference expression profile each have values representing the expression level of at least one biomarker selected from RPL22, VEGFA, G0S2, NES, TNFRSF25, DKFZP586P0123, COL8A2, ZNF3, RIPK5, RNFT2, ARHGEF12 and PTPN20A;
(c) selecting the biomarker reference expression profile most similar to the subject biomarker expression profile, to thereby predict a prognosis for the subject.

In some embodiments, the biomarker reference expression profile comprises a poor survival group or a good survival group.

In different embodiments, the at least one biomarker is any of two biomarkers, three biomarkers, four biomarkers, five biomarkers, six biomarkers, seven biomarkers, eight biomarkers, nine biomarkers, ten biomarkers, eleven biomarkers and twelve biomarkers.

In some embodiments, determining the biomarker expression level comprises use of quantitative PCR or an array, preferably a U133A chip.

In some embodiments, determining the biomarker expression profile comprises use of an antibody to detect polypeptide products of the biomarker.

In some embodiments, the sample comprises a tissue sample, preferably a sample suitable for immunohistochemistry.

According to a further aspect, there is provided a method of selecting a therapy for a subject with SQCC, comprising the steps:
(a) classifying the subject with SQCC into a poor survival group or a good survival group according to the method of any one of claims 1-19; and
(b) selecting adjuvant chemotherapy for the poor survival group or no adjuvant chemotherapy for the good survival group.

According to a further aspect, there is provided a method of selecting a therapy for a subject with SQCC, comprising the steps:
(a) determining the expression of at least one biomarker in a test sample from the subject selected from RPL22, VEGFA, G0S2, NES, TNFRSF25, DKFZP586P0123, COL8A2, ZNF3, RIPK5, RNFT2, ARHGEF12 and PTPN20A;
(b) comparing the expression of the at least one biomarker in the test sample with the same biomarker in a control sample;
(c) classifying the subject in a poor survival group or a good survival group, wherein a difference or a similarity in the expression of the at least three biomarkers between the control sample and the test sample is used to classify the subject into a poor survival group or a good survival group;
(d) selecting adjuvant chemotherapy if the subject is classified in the poor survival group and selecting no adjuvant chemotherapy if the subject is classified in the good survival group.

According to a further aspect, there is provided a composition comprising a plurality of isolated nucleic acid sequences, wherein each isolated nucleic acid sequence hybridizes to:
(a) a RNA product of at least one of twelve genes: RPL22, VEGFA, G0S2, NES, TNFRSF25, DKFZP586P0123, COL8A2, ZNF3, RIPK5, RNFT2, ARHGEF12 and PTPN20A; and/or
(b) a nucleic acid complementary to a),
wherein the composition is used to measure the level of RNA expression of the genes.

According to a further aspect, there is provided an array comprising, for each of at least one of twelve genes: RPL22, VEGFA, G0S2, NES, TNFRSF25, DKFZP586P0123, COL8A2, ZNF3, RIPK5, RNFT2, ARHGEF12 and PTPN20A, one or more polynucleotide probes complementary and hybridizable to an expression product of the gene.

According to a further aspect, there is provided a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out a method described herein.

According to a further aspect, there is provided a computer implemented product for predicting a prognosis or classifying a subject with SQCC comprising:
(a) a means for receiving values corresponding to a subject expression profile in a subject sample; and
(b) a database comprising a reference expression profile associated with a prognosis, wherein the subject biomarker expression profile and the biomarker reference profile each have at least three values representing the expression level of at least one biomarker selected from RPL22, VEGFA, G0S2, NES, TNFRSF25, DKFZP586P0123, COL8A2, ZNF3, RIPK5, RNFT2, ARHGEF12 and PTPN20A;
wherein the computer implemented product selects the biomarker reference expression profile most similar to the subject biomarker expression profile, to thereby predict a prognosis or classify the subject.

Preferably, a computer implemented product described herein is for use with a method described herein.

According to a further aspect, there is provided a computer implemented product for determining therapy for a subject with SQCC comprising:
(a) a means for receiving values corresponding to a subject expression profile in a subject sample; and
(b) a database comprising a reference expression profile associated with a therapy, wherein the subject biomarker expression profile and the biomarker reference profile each have at least one value, the at least one value representing the expression level of at least one biomarker selected from RPL22, VEGFA, G0S2, NES, TNFRSF25, DKFZP586P0123, COL8A2, ZNF3, RIPK5, RNFT2, ARHGEF12 and PTPN20A;
wherein the computer implemented product selects the biomarker reference expression profile most similar to the subject biomarker expression profile, to thereby predict the therapy.

According to a further aspect, there is provided a computer readable medium having stored thereon a data structure for storing a computer implemented product described herein.

Preferably, the data structure is capable of configuring a computer to respond to queries based on records belonging to the data structure, each of the records comprising:
(a) a value that identifies a biomarker reference expression profile of at least one gene selected from RPL22, VEGFA, G0S2, NES, TNFRSF25, DKFZP586P0123, COL8A2, ZNF3, RPM, RNFT2, ARHGEF12 and PTPN20A;
(b) a value that identifies the probability of a prognosis associated with the biomarker reference expression profile.

According to a further aspect, there is provided a computer system comprising
(a) a database including records comprising a biomarker reference expression profile of at least one gene selected from RPL22, VEGFA, G0S2, NES, TNFRSF25, DKFZP586P0123, COL8A2, ZNF3, RIPK5, RNFT2, ARHGEF12 and PTPN20A associated with a prognosis or therapy;
(b) a user interface capable of receiving a selection of gene expression levels of the at least one gene for use in comparing to the biomarker reference expression profile in the database;
(c) an output that displays a prediction of prognosis or therapy according to the biomarker reference expression profile most similar to the expression levels of the at least one gene.

According to a further aspect, there is provided a kit to prognose or classify a subject with early stage SQCC, comprising detection agents that can detect the expression products of at least one biomarker selected from RPL22, VEGFA, G0S2, NES, TNFRSF25, DKFZP586P0123, COL8A2, ZNF3, RIPK5, RNFT2, ARHGEF12 and PTPN20A, and instructions for use.

According to a further aspect, there is provided a kit to select a therapy for a subject with SQCC, comprising detection agents that can detect the expression products of at least one biomarker selected from RPL22, VEGFA, G0S2, NES, TNFRSF25, DKFZP586P0123, COL8A2, ZNF3, RIPK5, RNFT2, ARHGEF12 and PTPN20A, and instructions for use.

A person skilled in the art will appreciate that a number of detection agents can be used to determine the expression of the biomarkers. For example, to detect RNA products of the biomarkers, probes, primers, complementary nucleotide sequences or nucleotide sequences that hybridize to the RNA products can be used. To detect protein products of the biomarkers, ligands or antibodies that specifically bind to the protein products can be used.

Accordingly, in one embodiment, the detection agents are probes that hybridize to the at least 1 of the 12 biomarkers. A person skilled in the art will appreciate that the detection agents can be labeled.

The label is preferably capable of producing, either directly or indirectly, a detectable signal. For example, the label may be radio-opaque or a radioisotope, such as ³H, ¹⁴C, ³²P, ³⁵S; ¹²³I; ¹²⁵I; ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

The kit can also include a control or reference standard and/or instructions for use thereof. In addition, the kit can include ancillary agents such as vessels for storing or transporting the detection agents and/or buffers or stabilizers.

In a further aspect, the application provides computer programs and computer implemented products for carrying out the methods described herein. Accordingly, in one embodiment, the application provides a computer program product for use in conjunction with a computer having a processor and a memory connected to the processor, the computer program product comprising a computer readable storage medium having a computer mechanism encoded thereon, wherein the computer program mechanism may be loaded into the memory of the computer and cause the computer to carry out the methods described herein.

The advantages of the present invention are further illustrated by the following examples. The example and its particular details set forth herein are presented for illustration only and should not be construed as a limitation on the claims of the present invention.

### EXAMPLE

### Materials and Methods

Datasets: Four large, NSCLC, publicly available microarray datasets were used: 129 SQCC samples from Molecular Diagnostics, Veridex LLC (UM) (13), 85 NSCLC samples (44 SQCC and 41 ADC) samples from Duke University (Duke) (3), 138 NSCLC samples (76 SQCC and 62 ADC) from Sungkyunkwan University (SKKU) (7), and 327 ADC samples from the NCI Director's Challenge Consortium for the Molecular Classification of ADC (DCC) (11). UM was used as the training set, while the remaining three datasets served as independent test sets. In addition, qPCR validation of the signature was carried out in 62 SQCC samples from the University Health Network (UHN). Patient demographics of the five independent datasets are shown in Table 1. The primary survival endpoint was 5-year survival (in UM, Duke, DCC, and UHN where overall survival was used) or disease-free survival (SKKU).

Data pre-processing: The raw data of the Veridex dataset were made available by Dr. Mitch Raponi and the Veridex. Duke and DCC datasets were downloaded from http::Rdata.cgt.duke.edu/oncogene.php and https::Rcaarraydb.nci.nih.gov/caarray/publicExperimentDetailAction.do?expId=1015945236141280, respectively. Raw .cel files were pre-processed by the Robust Multichip Average (RMA) algorithm using RMAexpress v0.5 (55), and then log 2 transformed. Probe sets were annotated using NetAffx v4.2 annotation tool (56). Affymetrix assigns five grades (A, B, C, E, and R) to classify the quality of their probe sets used in the GeneChip (56). Matching probe or Grade A annotations represents the best quality transcript assignments with at least 9 of the 11 probes in a probe set match a transcript mRNA or gene model sequence. Therefore only probe sets with ‘grade A’ annotation were used for signature optimization. The GCRMA normalized data and the limited clinical information from SKKU were downloaded directly from the NCBI GEO database (http::Rwww.ncbi.nlm.nih.gov/geo/) with the accession number GSE8894. The normalized data was standardized by Z-score transformation, which centered the expression level to mean zero and standard deviation of one (57). It is noteworthy that two methods were used for the calculation of the risk score. The first method was used in the signature optimization where the risk score was the product of Z-score weighted by the coefficient from the univariate survival analysis (58,59). The second method was used when PCA analysis was applied to the 12-gene signature, where the Z-score was first weighted by coefficient of each gene in each of the 4 selected principal components and the risk score was the sum of the scores of the 4 principal components weighted by their coefficients in the multivariate model (Table 4).

Univariate analysis: Overall survival (date of surgery to date of last follow-up or death) was used as the outcome endpoint. Follow-up was truncated at 5 years. The association of the expression of individual probe sets with 5-year overall survival was evaluated by Cox proportional hazards regression. An inclusion criterion of p<0.005 was set for pre-selecting the candidate probe sets chosen for signature optimization (22).

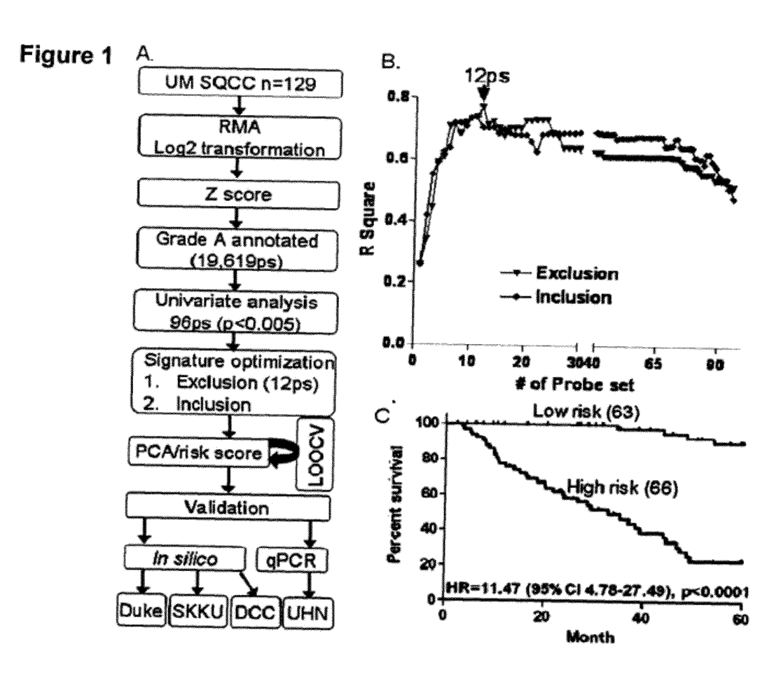

Signature selection: Signature optimization was conducted by an exclusion followed by an inclusion selection procedure (FIG. 1A). The exclusion procedure took all probe sets that met pre-selection criteria. Each probe set was excluded one at a time and a total risk score of the remaining probe sets was summed. The risk score was then dichotomized by an outcome-orientated optimization with cutoff procedures based on log-rank statistics (http::Rndc.mayo.edu/mayo/research/biostat/sasmacros.cfm) (60). The two resultant groups were introduced into the Cox proportional hazards model, where the Goodness-of-fit (R²) was calculated (61, 62). A probe set was excluded if its exclusion resulted in the largest R², or if multiple probe-sets had the same largest R², then the largest p-value of the two groups, or if multiple probe sets had the same largest p-value, then the largest univariate p value of the individual probe set. This procedure was repeated until there was only one probe set left. The inclusion procedure started with the probe set left by the exclusion procedure. Each probe set was added one at a time, the risk score of the included probe sets summed, the risk score dichotomized, and the R² of the Cox proportional hazards model calculated. The probe set was included once its inclusion resulted in the largest R², or if multiple probe-sets had the same largest R², then the smallest p-value of the two groups, or if multiple probe sets had the same smallest p-value, then the smallest univariate p-value of the individual probe-set. Finally, a set of minimum number of probe sets having the largest R² was identified as candidate in the gene signature.

Principal Component Analysis (PCA): To further reduce the data dimensionality and get rid of possible co-linearity expression of genes, PCA and multivariate Cox proportional hazards model with stepwise selection were used. PCA analysis identified 12 principal components (PC) and these PCs were introduced to a multivariate Cox proportional hazard model with stepwise selection using an inclusion criteria of 0.5 (sle=0.5). PCs who were significantly associated with survival (sls=0.05) retained. Four PCs were identified and their coefficients were listed in Table 4. The weight of each member of the 12-gene signature in each of the 4 PCs was listed in Table 4. Risk score was dichotomized at the optimal cutoff in the training set determined by the macro http::Rndc.mayo.edu/mayo/research/biostat/sasmacros.cfm (60). It gave a value of −0.056 as risk score cutoff (Table 4).

Leave-one-out-cross-validation (LOOCV): LOOCV was used as an internal validation of how accurate of the signature in assigning cases into low and high risk group. Cases were classified as low- or high-risk by the 12-gene signature based on the optimal cutoff in the entire cohort (n=129). Each case was then excluded once at a time and the class of low or high risk of the excluded case was predicted by the remaining cases (n=128). If the case was classified as high/low risk in the entire cohort but was assigned as low/high risk in the LOOCV, then it was an error. The acceptable predicting error rate was <5%.

In silico validation of expression signature: in silico validation of the prognostic signature was carried out separately on the 3 validation datasets form Duke (52), SKKU (53), and DCC (54). Expression level was Z-score transformed and the risk score was generated using the parameters listed in Table 5. Multivariate analysis was performed by Cox proportional hazards regression with the adjustment for stage, age and sex. Statistical analyses were performed using SAS v9.1 (SAS Institute, CA).

Quantitative-RT-PCR (qPCR) validation of the signature: qPCR validation was carried out in 62 SQCC samples from the University Heath Network. The patients did not receive any chemo- or radiotherapy before the samples were surgically resected. PrimerExpress v3.0 (AppliedBiosystems, Foster city, CA) was used to design primers. Primers were primarily designed within the target sequence of the probe sets, but once no primer could be found in this area, primers were designed in the CDS of the target gene. Primers used for quantification of the target genes were listed in Table 5. Five ng of cDNA was used for each reaction in the HT-7900 fast real-time PCR system (AppliedBiosystems, Foster city, CA). PCR reaction optimization was described previously (57). Four house-keeping genes (ACTB, TBP, BAT1, and B2M) were used initially (57); however, NormFinder (63) found that the combination of 3 genes (ACTB, TBP, and BAT1) was most stable (smallest variation, Table 6). Therefore, the mean of the Cts of the 3 house-keeping genes was used to normalize qPCR data. Expression was quantitated using 2^{-ΔΔCt} method and then Z-score transformed. Risk score was then calculated using the parameters listed in Table 4.

Protein-protein interaction (PPI) network construction and analysis: To determine the relationships among the proteins corresponding to the 12-gene SQCC prognostic signature and two published SQCC prognostic signatures [50-gene of Sun et al. (64) and 50-gene of Raponi et al. (51)], gene identifiers (EntrezGene IDs) and protein identifiers (SwissProt IDs) corresponding to the probe-sets of each of the prognostic signatures were obtained from NetAffx (NA24) annotation tables. The 12-gene signature mapped to 12 genes (Table 6), Sun's 50-gene signature mapped to 42 genes, while Raponi's 50-gene signature mapped to 48 genes, respectively. Protein-protein interaction (PPI) data were obtained by querying the Interologous Interaction Database (I²D v1.71; http::Rophid.utoronto.ca/i2d (65)). Interactions were obtained for 8/12 genes, 31/42, and 35/48 for signatures of our 12-gene, Sun's 50-gene and Raponi's 50-gene, respectively, including 8/9 genes overlapping between the latter two 50-gene signatures. The interacting proteins were then used to query the same database to determine whether any interactions are present among them. The resulting PPI network based on these three SQCC prognostic signatures comprised 1,075 nodes/proteins and 14,651 edges/interactions. The PPI network was visualized and annotated using NAViGaTOR v2.08 (http::Rophid.utoronto.ca/navigator/) (66).

Gene Ontology (GO) term and KEGG pathways enrichment analysis: GoStat (67) was used to evaluate GO term representation enrichment in the 12-gene signature. Significance was tested using Fisher's exact test and corrected by Benjamini and Hochberg method. For KEGG pathways (68) (http::Rwww.genome.jp/kegg/) representation enrichment analysis, Fisher's exact test was employed and the significance was corrected by the Bonferroni method. KEGG pathways representation enrichment in the protein-protein interaction (PPI) network of the three signature probe sets was also tested. PPI data was determined by testing KEGG pathway genes proportions (of 45 KEGG pathways for which at least 25% of the pathway genes were mapped in the experimentally determined PPI network) against expected proportions estimated from 1,000 randomly-generated PPI networks obtained by querying I²D using the same number of proteins in the interaction network of these 3 signatures (66 genes/proteins). Student's t-test was then used to compare the proportion in the experimentally determined PPI network against the distributions in random networks (69). The p-values were corrected by the Bonferroni method.

### Results

### New Prognostic Gene Expression Signature for Lung SQCC

The steps leading to signature identification and subsequent validation are represented schematically in FIG. 1A. In total there were 22,215 probe-sets (ps) on the U133A chip, 19,619 with grade A annotation. Univariate analysis identified 96 ps that were significantly associated with overall survival at p<0.005. The exclusion selection procedure started with these 96 ps and by stepwise exclusion, probe set 211514_at was identified as its last one.

This is followed by the inclusion procedure using 211514_at as its starting probe-set. The procedure included one probe-set at a time until all 96 ps were included. The exclusion procedure identified the largest R² of 0.77 with a combination of 12 ps (12-gene) (FIG. 1B). PCA analysis and the multivariate Cox proportional hazard model with stepwise selection revealed that 4 PCs were significantly associated with survival at p<0.05 (Table 4). Subsequent LOOCV identified a predicting error of the signature being 4.7% (6 cases). Thus, the 12-gene combination was established as the prognostic gene signature (Table 3).

When the risk score was dichotomized at the optimal cutoff (−0.056, Table 4), the 12-gene signature classified 63 and 66 SQCC patients into low- and high-risk groups, respectively with a significant difference in overall survival (HR=11.47, 95% CI 4.78-27.49, p<0.0001, FIG. 1C). Multivariate analysis revealed that the signature was an independent prognostic factor after adjusted for stage, age and sex (HR=15.18, 95% CI 6.04-38.11, p<0.0001, Table 7).

### In Silico Validation of the New 12-Gene Signature

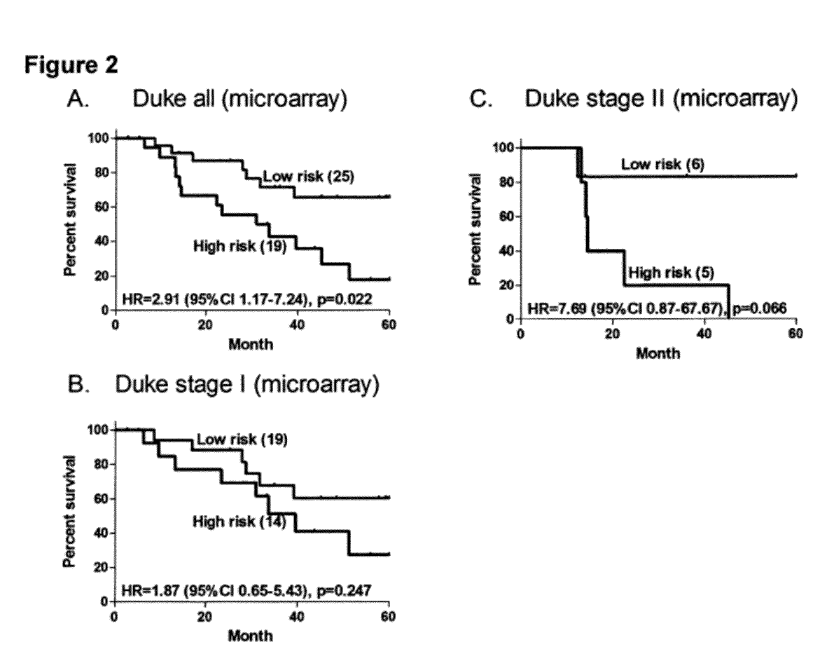

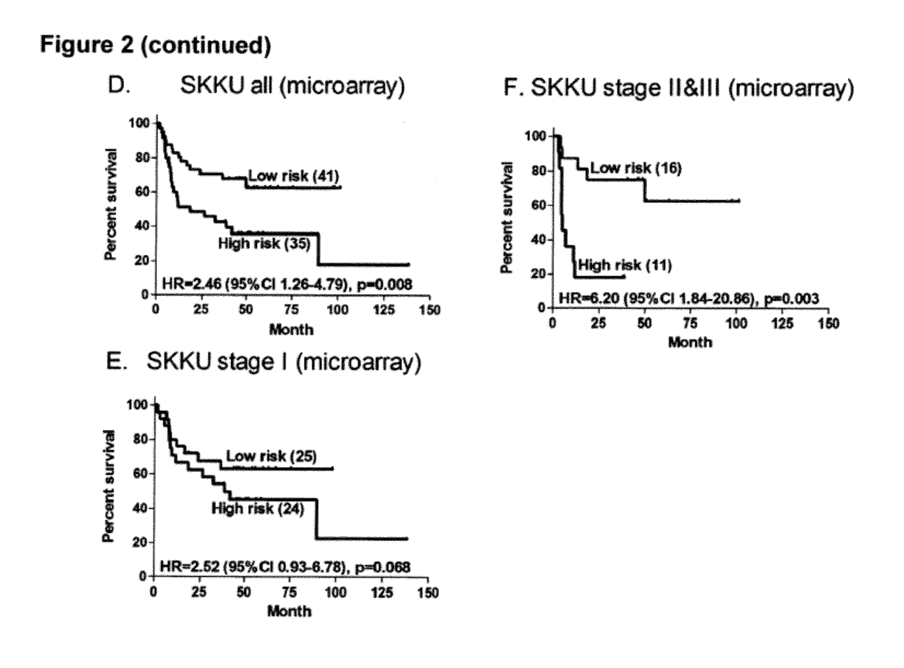

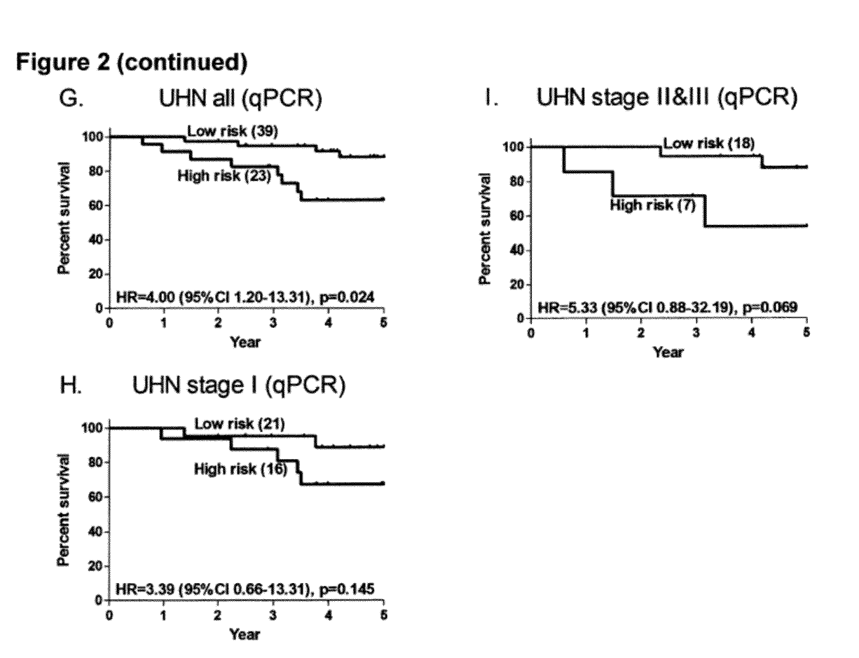

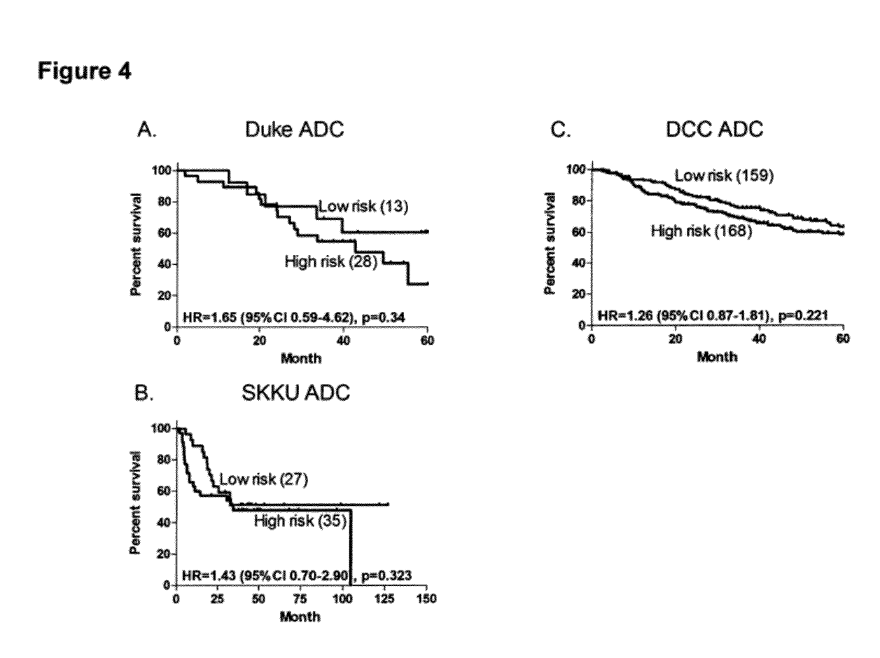

We first tested the 12-gene signature in the Duke 89 NSCLC dataset (46 SQCC and 43 ADC). Four patients with stage III-IV (2 ADC and 1 SQCC in stage III and 1 SQCC in stage IV) were excluded from further analysis (Table 1). When the risk score was dichotomized at −0.056, the signature classified 25 and 19 of 44 SQCC and 13 and 28 of 41 ADC into low- and high-risk groups, respectively. High-risk SQCC had significantly poorer survival than the low-risk group (HR=2.91, 95% CI 1.17-7.24, p=0.022, FIG. 2A), while the survival difference between the different risk groups for the ADC patients was not significant (HR=1.87, 95% CI 0.92-3.82, p=0.54, FIG. 4A). Stratified analysis by stage showed that the high risk-group classified by the signature had poorer survival in both stage I (HR=1.87, 95% CI 0.65-5.43, p=0.247, FIG. 2B) and II SQCC (HR=7.69, 95% CI 0.87-67.67, p=0.066, FIG. 2C). Furthermore, multivariate analysis showed that the signature was an independent prognostic factor in SQCC (HR=3.05, 95% CI 1.14-8.21, p=0.027) but not in ADC (HR=1.73, 95% CI 0.59-5.12, p=0.322, Table 2) after adjustment for stage, age and sex.

The SKKU dataset (7) included 138 stage I-III NSCLC (76 SQCC and 62 ADC) patients profiled using U133 plus 2 chip. This is the only NSCLC microarray dataset from Asia. Validation of our signature used recurrence-free survival as this is the only endpoint reported for this study. Because the GEO database has no raw data, we downloaded the expression data which was already GCRMA-preprocessed and log 2-transformed. Gene expression level was Z-score transformed and risk score was derived using the formula listed in Table 4. The 12-gene signature classified 41 and 35 of 76 SQCC and 27 and 35 of 62 ADC into low- and high-risk groups, respectively. Significantly shortened recurrence-free survival was observed in the high-risk group in the SQCC (HR=2.46, 95% CI 1.26-4.79, p=0.008, FIG. 2B) but not in the ADC (HR=1.43, 95% CI 0.70-2.90, p=0.323, FIG. 4B). Stratified analysis by stage showed that the signature worked in stage I (HR=2.52, 95% CI 0.93-6.78, p=0.068, FIG. 2E) and stage II and III (HR=6.20, 95% CI 1.84-20.86, p=0.003, FIG. 2F). Multivariate analysis showed that the signature was independent prognostic in SQCC (HR=2.77, 95% CI 1.34-5.73, p=0.006) but not in ADC (HR=1.92, 95% CI 0.91-4.05, p=0.086, Table 2) after adjustment for stage, age and sex.

To determine further whether the signature was prognostic in ADC, the 12-gene signature was tested in the largest available ADC microarray dataset from the NIH Director's Challenge Consortium study (11), which included 442 samples. Among them, 327 patients did not receive any adjuvant chemotherapy or radiotherapy and had follow-up longer than 1 month. The 12-gene signature was not prognostic (HR=1.26, 95% CI 0.87-1.81, p=0.221, FIG. 4C). Multivariate analysis showed that it was not an independent prognostic factor in ADC (HR=1.23, 95% CI 0.85-1.78, p=0.267, Table 2). These data confirm that the signature was not prognostic in ADC.

qPCR Validation in UHN SQCC Cohort

qPCR validation of the 12-gene signature was performed in an independent set of 62 snap-frozen SQCC samples from UHN. Fold change was calculated using 2^{-ΔΔCt} method and then Z-score transformed. Risk score was generated using parameters listed in Table 4. When risk score was dichotomized at −0.056, the 12-gene signature was able to separate 41 and 21 SQCC into low and high risk group with significant difference in 5-year overall survival (HR=4.00, 95% CI 1.20-13.31, p=0.024, FIG. 2G). Stratified analysis by stage revealed that the signature was able to separate low- and high-risk groups with different survival outcomes; however, the significance was marginal due to the small sample size (Stage I: HR=3.39, 95% CI 0.66-17.47, p=0.145, FIG. 2H and stage II&III: HR=5.33, 95% CI 0.88-32.19, p=0.069, FIG. 2I). Nevertheless, multivariate analysis again showed that the signature was an independent prognostic factor (HR=3.76, 95% CI 1.10-12.87, p=0.035, Table 2).

### The Composition of the 12-Gene Signature

Table 3 shows the members of 12-gene signature and their ranks of expression level, variance, and significance in the Veridex dataset (in decreasing order of importance). Notably, the expression level of individual genes varies greatly, from very high levels as for RPL22 (rank in the top 0.6%) to extremely low levels for PTPN20A/B (ranked at 99.7%). The standard deviation value also varies greatly, from very large as for G0S2 (rank at 1.9% of the total) to very small for RIPK5 (rank at 97.5% of the total). These data showed that the low-expression and low-variabity genes were as important as those with higher expression and higher variability.

Gene ontology (GO) (29) and KEGG pathways (26, 30) annotations revealed the involvement of several of the prognostic genes in signal transduction (e.g., VEGFA, TNFRSF25), cell cycle (e.g., VEGFA, G0S2), apoptosis (e.g., TNFRSF25), adhesion (e.g., COL8A2), transcription and translation (ZNF3 and RPL22, respectively) (Table 9)

### Protein-Protein Interaction Network Analysis

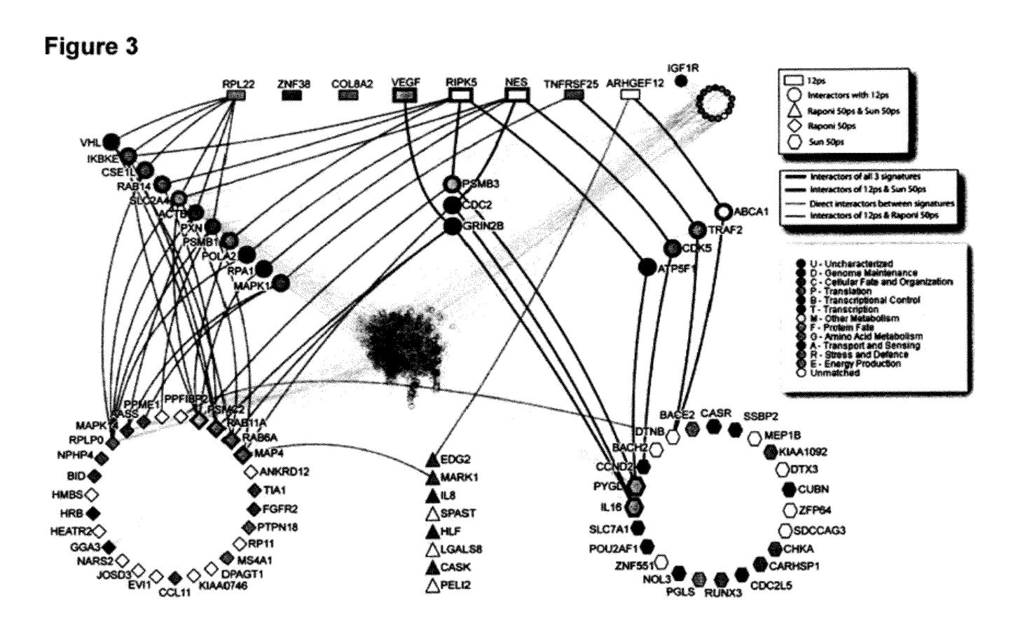

To assess the potential SQCC-specific biological relevance of the 12-gene signature genes further, we evaluated the functional relationship between our 12-gene signature and the reported Raponi (13) and Sun (8) 50-gene signatures (mapped to 12, 48 and 42 genes, respectively) through their corresponding protein-protein interaction (PPI) networks. We mapped 8/12 genes of the 12-gene signature, 35/48 and 31/42 for the Raponi and Sun signatures, respectively, to PPIs in the Interologous Interaction Databasever 1.7 (I²D; (23)). While the Raponi and Sun signatures have 10 overlapping probe sets (9 genes), the 12-gene signature has no probe sets/genes overlapping with either of the 50-gene signatures. However, direct interactions between the signature genes/proteins or via shared interacting proteins were seen among these signatures, implying a rich shared functional milieu (FIG. 3). Annotation of the resulting PPI network with KEGG pathways indicated significant enrichment for proteins from the MAPK signaling pathway (p=0.019; 80/1,075 proteins), which form direct interactions with 3, 14 and 9 genes/proteins of our, the Raponi and Sun signatures, respectively (Table 9, 10 and 11).

### DISCUSSION

We describe here the MAximizing R Square Algorithm (MARSA), a heuristic signature selection method that includes only genes contributing to the separation ability of the signature. By applying the algorithm to the UM dataset, we identified a 12-gene prognostic signature. The prognostic value of the 12-gene signature was validated in silico in 2 independent SQCC microarray datasets (Duke: HR=3.05, 95% CI 1.14-8.21, p=0.027; SKKU: HR=2.73, 95% CI 1.32-5.64, p=0.007, Table 2) but not in the corresponding ADC datasets (Table 2). Further, we confirmed the absence of the prognostic value of the 12-gene signature in the largest available ADC dataset from DCC containing 442 ADC samples (Table 2). Importantly, qPCR validation in another independent cohort confirmed that the signature was an independent prognostic factor in SQCC (Table 2). Combined, our data strongly suggested that the 12-gene signature is a valuable prognostic factor for SQCC.

The cellular origin and pathogenesis of SQCC and ADC remain controversial. In contrast to ADC, SQCC tends to arise in the epithelium of large airways and its etiology is clearly linked to smoking, suggesting different pathogenetic differences between the two lung cancer types (31). This is supported by differences in the occurrence of key genetic alterations in the two types of cancer (32). While frequently mutated in ADC, KRAS (33, 34) and EGFR (35) mutations occur very infrequently in SQCC. In contrast, P53 mutation (34), TIMP3 (36) and HIF-1α (37) overexpressions occur more frequently in SQCC than ADC of the lung. Moreover, gene expression profiling has demonstrated distinctive patterns among the subtypes of NSCLC (38). Additionally, target therapy indicates that significantly more ADC benefit from gifitinib and erlotinib treatments (39), Both treatments target EGFR, whereas SQCC benefit more from vandetanib (40), which targets both EGFR and VEGFR. Therefore, it may not be surprising that there could be gene signatures that are prognostic in SQCC but not in ADC patients.

Cancer phenotype is characterized by underlying gene expression. Thus gene expression signatures may predict clinical outcome. The fact that our signature had been validated consistently in multiple independent SQCC cohorts supports a notion that it might have captured a key gene expression program in squamous cancer biology. Indeed, many members of the 12-gene signature have been reported to be involved in processes underlying tumorigenesis, including: tumor necrosis factor receptor superfamily, member 25 (TNFRSF25), triggering apoptosis and activating the transcription factor NF-kappa-B in HEK293 or HeLa cells (41), RIPK5, a cell death inducer (42). Vascular endothelial growth factor (VEGF or VEGFA) has been extensively studied (43) and is a major regulator of tumor angiogenesis (44). ARHGEF4 (Rho guanine nucleotide exchange factor 4) is involved in G-protein mediated signaling, which has been implicated in regulating cell morphology and invasion (45). It has also been shown to interact directly with insulin-like growth factor receptor 1 (IGF1r), providing a link between G protein-coupled and IGF1r signaling pathways (46) (FIG. 3). Inhibitors of IGF1r are being studied in clinical trials in combination with chemotherapy and EGFR therapy, and preliminary result demonstrate high response rates in advanced NSCLC patients, especially of the SQCC subtype (47). In addition, our PPI analysis reveal significant enrichment in representation of genes involved in the MAPK signaling pathway (p=0.019), which has been shown as active in SQCC (48-50). These support the functional relevance of the 12-gene signature in SQCC. However, further biological and clinical validation of the signature is warranted.

Previous approaches to the identification of prognostic signatures filtered out low-expression or low-variance genes prior to signature selection. However, this might lead to the exclusion of low expression but important genes in the signatures. In fact, one third of the genes (ARHGEF12, RIPK5, PTPN20A, and ZNF3) in the 12-gene signature had expression levels in the lowest 20% (from 79.9-99.7%), while their variation (SD) was in the lowest 10% (from 91.5-97.5%, Table 3) of all probe-sets. The consistent performance of the 12-gene signature in the training and test cohorts implied that these low-expressed and low-variable genes might have played important roles in tumor progression, and thus these genes must be included in signature selection.

In summary, MARSA is an effective approach to identify prognostic gene expression signatures and this novel 12-gene prognostic signature appears specific for SQCC.

Although preferred embodiments of the invention have been described herein, it will be understood by those skilled in the art that variations may be made thereto without departing from the spirit of the invention or the scope of the appended claims. All documents mentioned herein, including but not limited to the following reference list, are hereby incorporated by reference.

### REFERENCE LIST

1. Ramaswamy S, Tamayo P, Rifkin R, et al. Multiclass cancer diagnosis using tumor gene expression signatures. Proc Natl Acad Sci USA 2001; 98:15149-54.
2. Tomida S, Koshikawa K, Yatabe Y, et al. Gene expression-based, individualized outcome prediction for surgically treated lung cancer patients. Oncogene 2004; 23:5360-70.
3. Potti A, Mukherjee S, Petersen R, et al. A genomic strategy to refine prognosis in early-stage non-small-cell lung cancer. N Engl J Med 2006; 355:570-80.
4. Chen H Y, Yu S L, Chen C H, et al. A five-gene signature and clinical outcome in non-small-cell lung cancer. N Engl J Med 2007; 356:11-20.
5. Lu Y, Lemon W, Liu P Y, et al. A gene expression signature predicts survival of patients with stage I non-small cell lung cancer. PLoS Med 2006; 3:e467.
6. Ikehara M, Oshita F, Sekiyama A, et al. Genome-wide cDNA microarray screening to correlate gene expression profile with survival in patients with advanced lung cancer. Oncol Rep 2004; 11:1041-4.
7. Lee E S, Son D S, Kim S H, et al. Prediction of Recurrence-Free Survival in Postoperative Non-Small Cell Lung Cancer Patients by Using an Integrated Model of Clinical Information and Gene Expression. Clin Cancer Res 2008; 14:7397-404.
8. Sun Z, Wigle D A, Yang P. Non-overlapping and non-cell-type-specific gene expression signatures predict lung cancer survival. J Clin Oncol 2008; 26:877-83.
9. Beer D G, Kardia S L, Huang C C, et al. Gene-expression profiles predict survival of patients with lung adenocarcinoma. Nat Med 2002; 8:816-24.
10. Bhattacharjee A, Richards W G, Staunton J, et al. Classification of human lung carcinomas by mRNA expression profiling reveals distinct adenocarcinoma subclasses. Proc Natl Acad Sci USA 2001; 98:13790-5.
11. Shedden K, Taylor J M, Enkemann S A, et al. Gene expression-based survival prediction in lung adenocarcinoma: a multi-site, blinded validation study. Nat Med 2008.
12. Larsen J E, Pavey S J, Passmore L H, Bowman R V, Hayward N K, Fong K M. Gene expression signature predicts recurrence in lung adenocarcinoma. Clin Cancer Res 2007; 13:2946-54.
13. Raponi M, Zhang Y, Yu J, et al. Gene expression signatures for predicting prognosis of squamous cell and adenocarcinomas of the lung. Cancer Res 2006; 66:7466-72.
14. Larsen J E, Pavey S J, Passmore L H, et al. Expression profiling defines a recurrence signature in lung squamous cell carcinoma. Carcinogenesis 2007; 28:760-6.
15. Bianchi F, Nuciforo P, Vecchi M, et al. Survival prediction of stage I lung adenocarcinomas by expression of 10 genes. J Clin Invest 2007; 117:3436-44.
16. Schumacher M, Binder H, Gerds T. Assessment of survival prediction models based on microarray data. Bioinformatics 2007; 23:1768-74.
17. Su A I, Cooke M P, Ching K A, et al. Large-scale analysis of the human and mouse transcriptomes. Proc Natl Acad Sci USA 2002; 99:4465-70.
18. Jongeneel C V, Iseli C, Stevenson B J, et al. Comprehensive sampling of gene expression in human cell lines with massively parallel signature sequencing. Proc Natl Acad Sci USA 2003; 100:4702-5.
19. Bolstad B M, Irizarry R A, Astrand M, Speed T P. A comparison of normalization methods for high density oligonucleotide array data based on variance and bias. Bioinformatics 2003; 19:185-93.
20. Affymetrix, editor. Transcript assignment for NetAffx™ annotation; 2006.
21. Lau S K, Boutros P C, Pintilie M, et al. Three-gene prognostic classifier for early-stage non small-cell lung cancer. J Clin Oncol 2007; 25:5562-9.
22. Simon R. Roadmap for developing and validating therapeutically relevant genomic classifiers. J Clin Oncol 2005; 23:7332-41.
23. Brown K R, Jurisica I. Unequal evolutionary conservation of human protein interactions in interologous networks. Genome Biol 2007; 8:R95.
24. Brown K R, Otasek D, Ali M, et al. NAViGaTOR: Network Analysis, Visualization and Graphing Toronto. Bioinformatics 2009; 25:3327-9.
25. Beissbarth T, Speed T P. GOstat: find statistically overrepresented Gene Ontologies within a group of genes. Bioinformatics 2004; 20:1464-5.
26. Kanehisa M, Goto S. KEGG: kyoto encyclopedia of genes and genomes. Nucleic Acids Res 2000; 28:27-30.
27. Larsen J E, Pavey S J, Bowman R, et al. Gene expression of lung squamous cell carcinoma reflects mode of lymph node involvement. Eur Respir J 2007; 30:21-5.
28. Roepman P, Jassem J, Smit E F, et al. An immune response enriched 72-gene prognostic profile for early-stage non-small-cell lung cancer. Clin Cancer Res 2009; 15:284-90.
29. Ashburner M, Ball C A, Blake J A, et al. Gene ontology: tool for the unification of biology. The Gene Ontology Consortium. Nat Genet 2000; 25:25-9.
30. Ogata H, Goto S, Sato K, Fujibuchi W, Bono H, Kanehisa M. KEGG: Kyoto Encyclopedia of Genes and Genomes. Nucleic Acids Res 1999; 27:29-34.
31. Ishikawa H, Nakayama Y, Kitamoto Y, et al. Effect of histologic type on recurrence pattern in radiation therapy for medically inoperable patients with stage I non-small-cell lung cancer. Lung 2006; 184:347-53.
32. Zhu C Q, Shih W, Ling C H, Tsao M S. Immunohistochemical markers of prognosis in non-small cell lung cancer: a review and proposal for a multiphase approach to marker evaluation. J Clin Pathol 2006; 59:790-800.
33. Salgia R, Skarin A T. Molecular abnormalities in lung cancer. J Clin Oncol 1998; 16:1207-17.
34. Tsao M S, Aviel-Ronen S, Ding K, et al. Prognostic and Predictive Importance of p53 and RAS for Adjuvant Chemotherapy in Non Small-Cell Lung Cancer. J Clin Oncol 2007; 25:5240-7.
35. Tsao M S, Sakurada A, Cutz J C, et al. Erlotinib in lung cancer—molecular and clinical predictors of outcome. N Engl J Med 2005; 353:133-44.
36. Mino N, Takenaka K, Sonobe M, et al. Expression of tissue inhibitor of metalloproteinase-3 (TIMP-3) and its prognostic significance in resected non-small cell lung cancer. J Surg Oncol 2007; 95:250-7.
37. Lee C H, Lee M K, Kang C D, et al. Differential expression of hypoxia inducible factor-1 alpha and tumor cell proliferation between squamous cell carcinomas and adenocarcinomas among operable non-small cell lung carcinomas. J Korean Med Sci 2003; 18:196-203.
38. Hofmann H S, Baffling B, Simm A, et al. Identification and classification of differentially expressed genes in non-small cell lung cancer by expression profiling on a global human 59.620-element oligonucleotide array. Oncol Rep 2006; 16:587-95.
39. Herbst R S, Fukuoka M, Baselga J. Gefitinib—a novel targeted approach to treating cancer. Nat Rev Cancer 2004; 4:956-65.
40. Heymach J V, Johnson B E, Prager D, et al. Randomized, placebo-controlled phase II study of vandetanib plus docetaxel in previously treated non small-cell lung cancer. J Clin Oncol 2007; 25:4270-7.
41. Marsters S A, Sheridan J P, Donahue C J, et al. Apo-3, a new member of the tumor necrosis factor receptor family, contains a death domain and activates apoptosis and NF-kappa B. Curr Biol 1996; 6:1669-76.
42. Zha J, Zhou Q, Xu L G, et al. RIPS is a RIP-homologous inducer of cell death. Biochem Biophys Res Commun 2004; 319:298-303.
43. Leung D W, Cachianes G, Kuang W J, Goeddel D V, Ferrara N. Vascular endothelial growth factor is a secreted angiogenic mitogen. Science 1989; 246:1306-9.
44. Folkman J. Angiogenesis in cancer, vascular, rheumatoid and other disease. Nat Med 1995; 1:27-31.
45. Kitzing T M, Sahadevan A S, Brandt D T, et al. Positive feedback between Dial, LARG, and RhoA regulates cell morphology and invasion. Genes Dev 2007; 21:1478-83.
46. Taya S, Inagaki N, Sengiku H, et al. Direct interaction of insulin-like growth factor-1 receptor with leukemia-associated RhoGEF. J Cell Biol 2001; 155:809-20.
47. Karp D D, Paz-Ares L G, Novello S, et al. High activity of the anti-IGF-IR antibody CP-751,871 in combination with paclitaxel and carboplatin in squamous NSCLC. J Clin Oncol 2008; 26 (suppl.).
48. Sekido Y, Fong K M, Minna J D. Molecular genetics of lung cancer. Annu Rev Med 2003; 54:73-87.
49. Fong K M, Sekido Y, Gazdar A F, Minna J D. Lung cancer. 9: Molecular biology of lung cancer: clinical implications. Thorax 2003; 58:892-900.
50. Scagliotti G V, Selvaggi G, Novello S, Hirsch F R. The biology of epidermal growth factor receptor in lung cancer. Clin Cancer Res 2004; 10:4227s-32s.
51. Raponi M, Zhang Y, Yu J, et al. Gene expression signatures for predicting prognosis of squamous cell and adenocarcinomas of the lung. Cancer Res 2006; 66:7466-72.
52. Potti A, Mukherjee S, Petersen R, et al. A genomic strategy to refine prognosis in early-stage non-small-cell lung cancer. N Engl J Med 2006; 355:570-80.
53. Lee E S, Son D S, Kim S H, et al. Prediction of Recurrence-Free Survival in Postoperative Non-Small Cell Lung Cancer Patients by Using an Integrated Model of Clinical Information and Gene Expression. Clin Cancer Res 2008; 14:7397-404.
54. Shedden K, Taylor J M, Enkemann S A, et al. Gene expression-based survival prediction in lung adenocarcinoma: a multi-site, blinded validation study. Nat Med 2008.
55. Bolstad B M, Irizarry R A, Astrand M, Speed T P. A comparison of normalization methods for high density oligonucleotide array data based on variance and bias. Bioinformatics 2003; 19:185-93.
56. Affymetrix, editor. Transcript assignment for NetAffx™ annotation; 2006.
57. Lau S K, Boutros P C, Pintilie M, et al. Three-gene prognostic classifier for early-stage non small-cell lung cancer. J Clin Oncol 2007; 25:5562-9.
58. Chen H Y, Yu S L, Chen C H, et al. A five-gene signature and clinical outcome in non-small-cell lung cancer. N Engl J Med 2007; 356:11-20.
59. Beer D G, Kardia S L, Huang C C, et al. Gene-expression profiles predict survival of patients with lung adenocarcinoma. Nat Med 2002; 8:816-24.
60. Mandrekar J N, Mandrekar S J, Cha S S. Cutpoint Determination Methods in Survival Analysis using SAS. SAS SUGI proceedings 2002; SUGI 28:261-28.
61. Kent J, O'Quigley J. Measures of dependence for censored survival data. Biometrika 1988; 75:525-34.
62. Heinzl H. Using SAS to calculate the Kent and O'Quigley measure of dependence for Cox proportional hazards regression model. Comput Methods Programs Biomed 2000; 63:71-6.
63. Andersen C L, Jensen J L, Orntoft T F. Normalization of real-time quantitative reverse transcription-PCR data: a model-based variance estimation approach to identify genes suited for normalization, applied to bladder and colon cancer data sets. Cancer Res 2004; 64:5245-50.
64. Sun Z, Wigle D A, Yang P. Non-overlapping and non-cell-type-specific gene expression signatures predict lung cancer survival. J Clin Oncol 2008; 26:877-83.
65. Brown K R, Jurisica I. Unequal evolutionary conservation of human protein interactions in interologous networks. Genome Biol 2007; 8:R95.
66. Brown K R, Otasek D, Ali M, et al. NAViGaTOR: Network Analysis, Visualization and Graphing Toronto. Bioinformatics 2009; 25:3327-9.
67. Beissbarth T, Speed T P. GOstat: find statistically overrepresented Gene Ontologies within a group of genes. Bioinformatics 2004; 20:1464-5.
68. Kanehisa M, Goto S. KEGG: kyoto encyclopedia of genes and genomes. Nucleic Acids Res 2000; 28:27-30.
69. Gortzak-Uzan L, Ignatchenko A, Evangelou A I, et al. A proteome resource of ovarian cancer ascites: integrated proteomic and bioinformatic analyses to identify putative biomarkers. J Proteome Res 2008; 7:339-51.

TABLE 1
Demographic data for patients in the five datasets

| | | UM | | Duke | | SKKU | | DCC* | | UHN | |
|---|---|---|---|---|---|---|---|---|---|---|---|
| n | | 129 | | 89 | | 138 | | 327 | | 62 | |
| Age | <65 | 52 | (40.3) | 33 | (37.1) | 79 | (57.2) | 152 | (46.5) | 20 | (32.3) |
| | ≧65 | 77 | (59.7) | 56 | (62.9) | 59 | (42.8) | 175 | (53.5) | 42 | (67.7) |
| Sex | Male | 82 | (63.6) | 54 | (60.7) | 104 | (75.4) | 172 | (52.6) | 41 | (66.1) |
| | Female | 47 | (36.4) | 35 | (39.3) | 34 | (24.6) | 155 | (47.4) | 21 | (33.9) |
| Stage | IA | 21 | (20.9) | 37 | (41.6) | 16 | (11.6) | 108 | (33.0) | 12 | (19.4) |
| | IB | 46 | (35.7) | 30 | (33.7) | 72 | (52.2) | 120 | (36.7) | 25 | (40.3) |
| | IIA | 6 | (4.7) | 5 | (5.6) | 6 | (4.3) | 17 | (5.2) | 4 | (6.5) |
| | IIB | 27 | (20.9) | 13 | (14.6) | 18 | (13.0) | 42 | (12.8) | 16 | (25.8) |
| | IIIA | 17 | (13.1) | 3** | (3.4) | 16 | (11.6) | 31 | (9.5) | 5 | (8.0) |

| | | | | | | | |
|---|---|---|---|---|---|---|---|
| IIIB | 6 | (4.7) | 10 | (7.2) | 8 | (2.4) | 0 |

| | IV | 0 | 1** | (1.1) | 0 | | 0 | | 0 |
|---|---|---|---|---|---|---|---|---|---|
| Histology | AD | 0 | 43 | (48.3) | 62 | (44.9) | 327 | (100) | 0 |

| | | | | | | | | | |
|---|---|---|---|---|---|---|---|---|---|
| SQ | 129 | (100) | 46 | (51.7) | 76 | (55.1) | 0 | 62 | (100) |

| | | | | | |
|---|---|---|---|---|---|
| Platform | U133A | U133 + 2 | U133 + 2 | U133A | qPCR |

UM: University of Michigan; SKKU: Sungkyunkwan University; DCC: Director's Challenge Consortium. The values represent number of patients and comparative percentage in bracket; U133 + 2: U133 plus 2; qPCR: quantitative-RT-PCR;
*1 case in DCC has no stage;
**not included in analysis.

TABLE 2
Validation of the 12-gene signature

| | Squamous cell carcinoma | | | | Adenocarcinoma | | | |
|---|---|---|---|---|---|---|---|---|
| | n | HR | 95% CI | p | n | HR | 95% CI | p |
| | In silico validation | | | | | | | |
| Duke | 44 | 3.05 | 1.14-8.21 | 0.027 | 43 | 1.73 | 0.59-5.12 | 0.322 |
| SKKU | 76 | 2.77 | 1.34-5.73 | 0.006 | 62 | 1.92 | 0.91-4.05 | 0.086 |
| DCC | | | | | 327 | 1.23 | 0.85-1.78 | 0.267 |
| | Quantitative-RT-PCR validation | | | | | | | |
| UHN | 62 | 3.76 | 1.10-12.87 | 0.035 | | | | |

The prognostic effect of the MARSA 12-gene signature was adjusted for stage, patients' age and sex; n, number of patients; HR: hazard ratio; 95% CI: 95% confidence interval; Duke, Duke University; SKKU, Sungkyunkwan University; DCC, Director's Challenge Consortium.

TABLE 3
Composition of the 12-gene signature

| | | | Rank of exp. | | Rank of SD | | Rank of sig. | |
|---|---|---|---|---|---|---|---|---|
| Probe Set | Gene Symbol | Gene Title | [n = 19619 (%)] | | [n = 19619 (%)] | | [n = 96 (%)] | |
| 221775_x_at | RPL22 | Ribosomal protein L22 | 117 | (0.6) | 12095 | (61.7) | 79 | (82.3) |
| 211527_x_at | VEGFA | Vascular endothelial | 3660 | (18.7) | 910 | (4.6) | 48 | (50.0) |
| | | growth factor A | | | | | | |
| 213524_s_at | G0S2 | G0/G1switch 2 | 4403 | (22.4) | 365 | (1.9) | 69 | (71.9) |
| 218678_at | NES | Nestin | 4504 | (23.0) | 4749 | (24.2) | 64 | (66.7) |
| 211282_x_at | TNFRSF25 | Tumor necrosis factor | 7582 | (38.7) | 6614 | (33.7) | 59 | (61.5) |
| | | receptor superfamily, | | | | | | |
| | | member 25 | | | | | | |
| 36552_at | DKFZP586P0123 | Hypothetical protein | 9094 | (46.4) | 11934 | (60.8) | 31 | (32.3) |
| 221900_at | COL8A2 | Collagen, type VIII, | 10236 | (52.2) | 1574 | (8.0) | 66 | (68.8) |
| | | alpha 2 | | | | | | |
| 219604_s_at | ZNF3 | Zinc finger protein 3 | 15673 | (79.9) | 18300 | (93.3) | 71 | (74.0) |
| 211514_at | RIPK5 | Receptor interacting | 15976 | (81.4) | 19129 | (97.5) | 2 | (2.1) |
| | | protein kinase 5 | | | | | | |
| 221909_at | RNFT2 | Ring finger protein, | 16306 | (83.1) | 2740 | (14.0) | 3 | (3.1) |
| | | transmembrane 2 | | | | | | |
| 201335_s_at | ARHGEF12 | Rho guanine nucleotide | 17123 | (87.3) | 18491 | (94.3) | 21 | (21.9) |
| | | exchange factor (GEF) 12 | | | | | | |
| 215172_at | PTPN20A/B | Protein tyrosine | 19558 | (99.7) | 17956 | (91.5) | 65 | (67.7) |
| | | phosphatase, non-receptor | | | | | | |
| | | type 20A/B | | | | | | |

Rank of exp.: rank of expression level (from high to low); Rank of SD: rank of standard deviation (from large to small); Rank of sig.: rank of significance level (from high to low).

TABLE 4
Coefficient of each gene in each principal component
and coefficient of each principal component

| Probe set | PC1 | PC2 | PC3 | PC10 |
|---|---|---|---|---|
| 201335_s_at | 0.296136 | 0.036644 | −0.07514 | −0.06007 |
| 211282_x_at | 0.372601 | −0.19435 | −0.1645 | 0.042215 |
| 211514_at | −0.12086 | −0.46083 | −0.19608 | 0.097768 |
| 211527_x_at | 0.113931 | −0.07118 | 0.597034 | −0.04887 |
| 213524_s_at | −0.04676 | 0.263985 | 0.469596 | −0.24413 |
| 215172_at | 0.227727 | 0.498903 | 0.070964 | 0.771239 |
| 218678_at | 0.074925 | 0.391389 | 0.078098 | −0.31993 |
| 219604_s_at | 0.440798 | −0.27243 | 0.088402 | 0.189042 |
| 221775_x_at | 0.301365 | −0.26519 | 0.208401 | 0.106245 |
| 221900_at | −0.33056 | 0.197833 | −0.34046 | 0.160601 |
| 221909_at | 0.418358 | 0.143587 | −0.27964 | −0.35111 |
| 36552_at | 0.341776 | 0.259564 | −0.30884 | −0.17263 |

Risk score = pc1*0.76657 + pc2*0.49732 + pc3*0.47963 + pc10* − 0.41455 Risk score cutoff (Low/High risk group): −0.056

TABLE 5
Primers used for qPCR validation

| | Seq Id | |
|---|---|---|
| Oligo sequence (5′ to 3′) | No. | Oligo name |
| TGACGCACCTGAAGATAACTTTG | 1 | ARHGEF12 F1 |
| GCACAGAAATGTTGGTATGTGAAGA | 2 | ARHGEF12 R1 |
| CGGCCACCCATCTGTCA | 3 | TNFRSF25 F1 |
| TCCAGCTGTTACCCACCAACT | 4 | TNFRSF25 R1 |
| TTGCTCAGAGCGGAGAAAGC | 5 | VEGFA F1 |
| CTTGCAACGCGAGTCTGTGT | 6 | VEGFA R1 |
| GGGTGGACTAACTTTGGACACAA | 7 | PTPN20 F1 |
| GAAATGCTTCCCAGACCAACA | 8 | PTPN20 R1 |
| CCAAGAATGGAGGCTGTAGGAA | 9 | NES F1 |
| GGATTCAGCTGACTTAGCCTATGAG | 10 | NES R1 |
| GGCTCCTGTGAAAAAGCTTGTG | 11 | RPL22 F1 |
| GGCAGCATCCATGATTCCAT | 12 | RPL22 R1 |
| ATGGGAGCCCACGGAACTA | 13 | COL8A2 F1 |
| AACCACCCCTCCTGAAAGGT | 14 | COL8A2 R1 |
| CCACGGATGCCTCAAGAGA | 15 | DKFZP586P0123F1 |
| CCACAGAAAAAAGGAGCTGAAATT | 16 | DKFZP586P0123R1 |
| AGCCTTGCCACAATCTTTGC | 17 | ZNF3 F1 |
| GTGGACCGGCCCTATGACT | 18 | ZNF3 R1 |
| GAGCCCACCTGCCATCACT | 19 | DSTYK F1 |
| CTATTGAGCCGAGTCCGGAAT | 20 | DSTYK R1 |
| AGAGCCCAGAGCCGAGATG | 21 | G0S2 F1 |
| ACGCTGCCCAGCACGTA | 22 | G0S2 R1 |
| TGGGCGGAGTTAGGAAAGC | 23 | RNFT2 F1 |
| GGAACTCGGCCTGACAGATG | 24 | RNFT2 R1 |

TABLE 6
Stability score of the house-keeping genes

| Gene name | Stability value |
|---|---|
| TBP | 0.565 |
| BAT1 | 0.376 |
| B2M | 0.952 |
| ACTB | 0.508 |
| mean of the 4 | 0.126 |
| mean of BAT1 and ACTB | 0.214 |
| mean of TBP, BAT1, and ACTB | 0.017 |

TABLE 7
Multivariate analysis in UM

| Variable | HR | 95% CI | p value |
|---|---|---|---|
| 12-gene signature | 15.18 | 6.04-38.11 | <.0001 |
| Stage II&III | 2.13 | 1.12-4.04 | 0.022 |
| Age ≧65 y | 0.79 | 0.42-1.50 | 0.478 |
| Female | 0.86 | 0.45-1.65 | 0.651 |

TABLE 9
GO terms and KEGG pathway annotation of the 12-gene signature genes

| | | Gene | Entrez | | | |
|---|---|---|---|---|---|---|
| Probeset ID | Gene Title | Symbol | Gene | GO Biological process | GO Cellular component | KEGG pathway |
| 201335_s_at | Rho guanine | ARHGEF12 | 23365 | regulation of Rho | intracellular, cytoplasm, | Axon guidance, |
| | nucleotide | | | protein signal | membrane | Regulation of actin |
| | exchange | | | transduction | | cytoskeleton |
| | factor (GEF) | | | | | |
| | 12 | | | | | |
| 211282_x_at | Tumor | TNFRSF25 | 8718 | apoptosis, apoptosis, | intracellular, cytosol, | Cytokine-cytokine |
| | necrosis | | | induction of apoptosis, | plasma membrane, integral | receptor |
| | factor | | | immune response, signal | to plasma membrane, | interaction |
| | receptor | | | transduction, cell | membrane, integral to | |
| | superfamily, | | | surface receptor linked | membrane | |
| | member 25 | | | signal transduction, | | |
| | | | | induction of apoptosis | | |
| | | | | by extracellular signals, | | |
| | | | | regulation of Rho | | |
| | | | | protein signal | | |
| | | | | transduction, regulation | | |
| | | | | of apoptosis, positive | | |
| | | | | regulation of I-kappaB | | |
| | | | | kinase/NF-kappaB | | |
| | | | | cascade | | |
| 211514_at | Receptor | RIPK5 | 25778 | protein amino acid | cytoplasm | |
| | interacting | | | phosphorylation | | |
| | protein | | | | | |
| | kinase 5 | | | | | |
| 211527_x_at | Vascular | VEGFA | 7422 | regulation of | proteinaceous extracellular | Cytokine-cytokine |
| | endothelial | | | progression through cell | matrix, extracellular space, | receptor |
| | growth factor | | | cycle, angiogenesis, | membrane | interaction, mTOR |
| | A | | | vasculogenesis, | | signaling pathway, |
| | | | | response to hypoxia, | | VEGF signaling |
| | | | | signal transduction, | | pathway, Focal |
| | | | | multicellular organismal | | adhesion, Renal |
| | | | | development, nervous | | cell carcinoma, |
| | | | | system development, | | Pancreatic cancer, |
| | | | | cell proliferation, | | Bladder cancer |
| | | | | positive regulation of | | |
| | | | | cell proliferation, cell | | |
| | | | | migration, cell | | |
| | | | | differentiation, positive | | |
| | | | | regulation of vascular | | |
| | | | | endothelial growth | | |
| | | | | factor receptor signaling | | |
| | | | | pathway, negative | | |
| | | | | regulation of apoptosis, | | |
| | | | | induction of positive | | |
| | | | | chemotaxis | | |
| 213524_s_at | G0/G1switch | G0S2 | 50486 | regulation of | NA | NA |
| | 2 | | | progression through cell | | |
| | | | | cycle, cell cycle | | |
| 215172_at | Protein | PTPN20A/B | 26095 | protein amino acid | cytoplasm, microtubule | |
| | tyrosine | | | dephosphorylation, | | |
| | phosphatase, | | | dephosphorylation | | |
| | non-receptor | | | | | |
| | type 20A/B | | | | | |
| 218678_at | Nestin | NES | 10763 | central nervous system | intermediate filament, | Cell |
| | | | | development | intermediate filament | Communication |
| 219604_s_at | Zinc finger | ZNF3 | 7551 | transcription, regulation | intracellular, nucleus | |
| | protein 3 | | | of transcription, DNA- | | |
| | | | | dependent, regulation of | | |
| | | | | transcription, DNA- | | |
| | | | | dependent, multicellular | | |
| | | | | organismal | | |
| | | | | development, cell | | |
| | | | | differentiation, | | |
| | | | | leukocyte activation | | |
| 221775_x_at | Ribosomal | RPL22 | 6146 | translation, translation | intracellular, ribosome, | |
| | protein L22 | | | | cytosolic large ribosomal | |
| | | | | | subunit (sensu Eukaryota), | |
| | | | | | ribonucleoprotein complex | |
| 221900_at | Collagen, | COL8A2 | 1296 | phosphate transport, cell | proteinaceous extracellular | |
| | type VIII, | | | adhesion, cell-cell | matrix, proteinaceous | |
| | alpha 2 | | | adhesion, extracellular | extracellular matrix, | |
| | | | | matrix organization and | basement membrane, | |
| | | | | biogenesis | cytoplasm | |
| 221909_at | Transmembrane | RNFT2 | 84900 | NA | membrane, integral to | |
| | protein 118 | | | | membrane | |
| 36552_at | Hypothetical | DKFZP586P | 26005 | NA | NA | NA |
| | protein | 0123 | | | | |

NA—Not available

TABLE 10
The 12-gene SQCC prognostic signature identifiers
(Probe set, Gene Symbol, Entrez Gene, SwissProt)

| Probe set | Gene Symbol | Entrez Gene | SwissProt |
|---|---|---|---|
| 201335_s_at | ARHGEF12 | 23365 | Q9NZN5* |
| 211282_x_at | TNFRSF25 | 8718 | Q93038* |
| 211514_at | RIPK5 | 25778 | Q6XUX3 |
| 211527_x_at | VEGFA | 7422 | P15692 |
| 213524_s_at | G0S2 | 50486 | P27469 |
| 215172_at | PTPN20A/B | 26095 | Q4JDL3 |
| 218678_at | NES | 10763 | P48681 |
| 219604_s_at | ZNF3 | 7551 | P17036 |
| 221775_x_at | RPL22 | 6146 | P35268* |
| 221900_at | COL8A2 | 1296 | P25067 |
| 221909_at | RNFT2 | 84900 | Q96SU5 |
| 36552_at | DKFZP586P0123 | 26005 | Q4AC94 |

SwissProt in boldface indicates protein is in PPI network (FIG. 3)
*Binds a protein in MAPK signaling pathway

TABLE 11
Raponi 50-gene SQCC prognostic signature identifiers
(Probe set, Gene Symbol, Entrez Gene, SwissProt)

| Probe set | Gene Symbol | Entrez Gene | SwissProt |
|---|---|---|---|
| 200863_s_at | RAB11A | 8766 | P62491* |
| 201033_x_at | LOC643779 | 6175 | P05388* |
| 201033_x_at | RPLP0 | 643779 | na |
| 201067_at | PSMC2 | 5701 | P35998* |
| 201448_at | TIA1 | 7072 | P31483 |
| 201449_at | TIA1 | 7072 | P31483 |
| 202530_at | MAPK14 | 1432 | Q16539* |
| 203040_s_at | HMBS | 3145 | P08397 |
| 203082_at | BMS1 | 9790 | Q14692 |
| 203196_at** | ABCC4 | 10257 | O15439 |
| 203545_at | ALG8 | 79053 | Q9BVK2 |
| 203555_at | PTPN18 | 26469 | Q99952 |
| 203638_s_at | FGFR2 | 2263 | P21802* |
| 204037_at** | EDG2 | 1902 | Q92633 |
| 204493_at | BID | 637 | P55957* |
| 204753_s_at** | HLF | 3131 | Q16534* |
| 205624_at | CPA3 | 1359 | P15088 |
| 207513_s_at | ZNF189 | 7743 | O75820 |
| 207620_s_at** | CASK | 8573 | O14936* |
| 208228_s_at | FGFR2 | 2263 | P21802* |
| 208856_x_at | LOC643779 | 6175 | P05388* |
| 208856_x_at | RPLP0 | 643779 | na |
| 208933_s_at** | LGALS8 | 3964 | O00214 |
| 208935_s_at** | LGALS8 | 3964 | O00214 |
| 209411_s_at | GGA3 | 23163 | Q9NZ52* |
| 209509_s_at | DPAGT1 | 1798 | Q9H3H5* |
| 209748_at** | SPAST | 6683 | Q9UBP0 |
| 210133_at | CCL11 | 6356 | P51671 |
| 210406_s_at | RAB6A | 5870 | P20340* |
| 210406_s_at | RAB6C | 84084 | Q9H0N0 |
| 210406_s_at | LOC150786 | 150786 | Q53S08 |
| 211596_s_at | LRIG1 | 26018 | Q96JA1 |
| 212286_at | ANKRD12 | 23253 | Q6UB98 |
| 212314_at | KIAA0746 | 23231 | Q68CR1 |
| 212841_s_at | PPFIBP2 | 8495 | Q8ND30 |
| 213471_at | NPHP4 | 261734 | O75161 |
| 214829_at | AASS | 10157 | Q9UDR5* |
| 217227_x_at** | IL8 | 3576 | P10145 |
| 217418_x_at | MS4A1 | 931 | P11836 |
| 217783_s_at | YPEL5 | 51646 | P62699 |
| 217841_s_at | PPME1 | 51400 | Q9Y570* |
| 218092_s_at | HRB | 3267 | P52594 |
| 218460_at | HEATR2 | 54919 | Q86Y56 |
| 218546_at | C1orf115 | 79762 | Q9H7X2 |
| 219132_at** | PELI2 | 57161 | Q9HAT8 |
| 219217_at | NARS2 | 79731 | Q96I59 |
| 219741_x_at | ZNF552 | 79818 | Q6P5A6 |
| 220285_at | FAM108B1 | 51104 | Q5VST7 |
| 221047_s_at** | MARK1 | 4139 | Q9P0L2* |
| 221580_s_at | JOSD3 | 79101 | Q9H5J8 |
| 221622_s_at | TMEM126B | 55863 | Q9NZ29 |
| 221884_at | EVI1 | 2122 | Q03112 |
| 243_g_at | MAP4 | 4134 | P27816 |
| 49077_at | PPME1 | 51400 | Q9Y570* |

SwissProt in boldface indicates protein is in PPI network (FIG. 3)
*Binds a protein in MAPK signaling pathway;
**Probe set found in Sun 50-gene;
NA: not available

TABLE 12
Sun 50-gene SQCC prognostic signature identifiers
(Probe set, Gene Symbol, Entrez Gene, SwissProt)

| Probe set | Gene Symbol | Entrez Gene | SwissProt |
|---|---|---|---|
| 200951_s_at | CCND2 | 894 | P30279 |
| 202746_at | ITM2A | 9452 | O43736 |
| 202747_s_at | ITM2A | 9452 | O43736 |
| 202990_at | PYGL | 5836 | P06737 |
| 203196_at** | ABCC4 | 10257 | O15439 |
| 203787_at | SSBP2 | 23635 | P81877 |
| 204037_at** | EDG2 | 1902 | Q92633 |
| 204197_s_at | RUNX3 | 864 | Q13761 |
| 204198_s_at | RUNX3 | 864 | Q13761 |
| 204266_s_at | CHKA/LOC650122 | 1119/650122 | P35790 |
| 204753_s_at** | HLF | 3131 | Q16534* |
| 204755_x_at | HLF | 3131 | Q16534* |
| 205267_at | POU2AF1 | 5450 | Q16633 |
| 206566_at | SLC7A1 | 6541 | P30825 |
| 206775_at | CUBN | 8029 | O60494 |
| 207028_at | MYCNOS | 10408 | P40205 |
| 207251_at | MEP1B | 4225 | Q16820* |
| 207620_s_at** | CASK | 8573 | O14936* |
| 208933_s_at** | LGALS8 | 3964 | O00214 |
| 208935_s_at** | LGALS8 | 3964 | O00214 |
| 209748_at** | SPAST | 6683 | Q9UBP0 |
| 209828_s_at | IL16 | 3603 | Q14005* |
| 210577_at | CASK | 846 | P41180* |
| 210965_x_at | CDC2L5 | 8621 | Q14004 |
| 211721_s_at | ZNF551 | 90233 | Q7Z340 |
| 212570_at | ENDOD1 | 23052 | O94919 |
| 213309_at | PLCL2 | 23228 | Q9UPR0 |
| 214253_s_at | DTNB | 1838 | O60941* |
| 215763_at | na | Na | na |
| 216147_at | na | Na | na |
| 216263_s_at | NGDN | 25983 | Q8NEJ9 |
| 217227_x_at** | IL8 | 3576 | P10145 |
| 217867_x_at | BACE2 | 25825 | Q9Y5Z0 |
| 218384_at | CARHSP1 | 23589 | Q9Y2V2 |
| 218388_at | PGLS | 25796 | O95336 |
| 218427_at | SDCCAG3 | 10807 | Q5SXN3 |
| 218507_at | HIG2 | 29923 | Q9Y5L2 |
| 219003_s_at | MANEA | 79694 | Q7Z3V7 |
| 219132_at** | PELI2 | 57161 | Q9HAT8 |
| 219536_s_at | ZFP64 | 55734 | Q9NPA5 |
| 219582_at | OGFRL1 | 79627 | Q5TC84 |
| 219659_at | ATP8A2 | 51761 | Q9NTI2 |
| 220692_at | na | Na | na |
| 220723_s_at | FLJ21511 | 80157 | Q9H720 |
| 221047_s_at** | MARK1 | 4139 | Q9P0L2* |
| 221234_s_at | BACH2 | 60468 | Q9BYV9* |
| 222048_at | na | Na | na |
| 49049_at | DTX3 | 196403 | Q8N9I9 |
| 59625_at | NOL3 | 8996 | O60936* |
| 65472_at | na | NA | NA |

SwissProt in boldface indicates protein is in PPI network (FIG. 3);
*binds a protein in MAPK signaling pathway;
**Probe set found in Raponi 50-gene;
NA: not available

1. A method of prognosing or classifying a subject with lung squamous cell carcinoma SQCC comprising:
(a) determining the expression of at least one biomarker in a test sample from the subject selected from RPL22, VEGFA, G0S2, NES, TNFRSF25, DKFZP586P0123, COL8A2, ZNF3, RIPK5, RNFT2, ARHGEF12 and PTPN20A; and
(b) comparing expression of the at least one biomarker in the test sample with expression of the at least one biomarker in a control sample;
wherein a difference or similarity in the expression of the at least one biomarker between the control and the test sample is used to prognose or classify the subject with SQCC into a poor survival group or a good survival group.

2. A method of predicting prognosis in a subject with lung squamous cell carcinoma (SQCC) comprising the steps:
(a) obtaining a subject biomarker expression profile in a sample of the subject;
(b) obtaining a biomarker reference expression profile associated with a prognosis, wherein the subject biomarker expression profile and the biomarker reference expression profile each have values representing the expression level of at least one biomarker selected from RPL22, VEGFA, G0S2, NES, TNFRSF25, DKFZP586P0123, COL8A2, ZNF3, RIPK5, RNFT2, ARHGEF12 and PTPN20A;
(c) selecting the biomarker reference expression profile most similar to the subject biomarker expression profile, to thereby predict a prognosis for the subject.

3. The method of claim 2, wherein the biomarker reference expression profile comprises a poor survival group or a good survival group.

4. The method of claim 2, wherein the at least one biomarker is two biomarkers.

5. The method of claim 2, wherein the at least one biomarker is three biomarkers.

6. The method of claim 2, wherein the at least one biomarker is four biomarkers.

7. The method of claim 2, wherein the at least one biomarker is five biomarkers.

8. The method of claim 2, wherein the at least one biomarker is six biomarkers.

9. The method of claim 2, wherein the at least one biomarker is seven biomarkers.

10. The method of claim 2, wherein the at least one biomarker is eight biomarkers.

11. The method of claim 2, wherein the at least one biomarker is nine biomarkers.

12. The method of claim 2, wherein the at least one biomarker is ten biomarkers.

13. The method of claim 2, wherein the at least one biomarker is eleven biomarkers.

14. The method of claim 2, wherein the at least one biomarker is twelve biomarkers.

15. The method of claim 2, wherein determining the biomarker expression level comprises use of quantitative PCR or an array.

16. The method of claim 15, wherein the array is a U133A chip.

17. The method of claim 2, wherein determining the biomarker expression profile comprises use of an antibody to detect polypeptide products of the biomarker.

18. The method of claim 17, wherein the sample comprises a tissue sample.

19. The method of claim 18, wherein the sample comprises a tissue sample suitable for immunohistochemistry.

20. A method of selecting a therapy for a subject with SQCC, comprising the steps:
(a) classifying the subject with SQCC into a poor survival group or a good survival group according to the method of claim 2; and
(b) selecting adjuvant chemotherapy for the poor survival group or no adjuvant chemotherapy for the good survival group.

21.-22. (canceled)

23. An array comprising, for each of at least one of twelve genes: RPL22, VEGFA, G0S2, NES, TNFRSF25, DKFZP586P0123, COL8A2, ZNF3, RIPK5, RNFT2, ARHGEF12 and PTPN20A, one or more polynucleotide probes complementary and hybridizable to an expression product of the gene. COL8A2, ZNF3, RIPK5, RNFT2, ARHGEF12 and PTPN20A, and instructions for use.

24-33. (canceled)
